(19) United States
(12) Patent Application Publication
(10) Pub. No.: US 2025/0114608 A1
(43) Pub. Date: Apr. 10, 2025

(54) PARAMETERIZING TECHNIQUES FOR SUB-PERCEPTION AND PARESTHESIA THERAPY

(71) Applicant: Boston Scientific Neuromodulation Corporation, Valencia, CA (US)

(72) Inventors: Andrew James Haddock, Los Angeles, CA (US); Rosana Esteller, Santa Clarita, CA (US)

(21) Appl. No.: 18/898,118

(22) Filed: Sep. 26, 2024

Related U.S. Application Data
(60) Provisional application No. 63/543,139, filed on Oct. 9, 2023.

(51) Int. Cl. A61N 1/36; A61N 1/05

(52) U.S. Cl. CPC A61N 1/36139; A61N 1/0551; A61N 1/36071; A61N 1/36132; A61N 1/36185

(57) ABSTRACT

A system for closed-loop control of sub-perception spinal cord stimulation therapy is provided. The system includes an implantable pulse generator (IPG) with an evoked compound action potential (ECAP) recording module and accelerometer. A regression model maps accelerometer data to optimal stimulation parameters for consistent nerve activation. This enables closed-loop therapy in the sub-perception domain where ECAP features are not visually detectable. A clinician programmer applies the model and sub-perception dose to therapy programs deployed to the IPG. The model can be customized to each patient by collecting ECAP and accelerometer data. The mapping reconciles ECAP-based control between perception and sub-perception domains by supplementing unavailable ECAP data with accelerometer data. This improves stimulation therapies for patients receiving sub-perception and multi-sensor paresthesia treatments.

### CLAIM OF PRIORITY

This application claims the benefit of U.S. Provisional Application No. 63/543,139, filed on Oct. 9, 2023, which is hereby incorporated by reference in its entirety.

### TECHNICAL FIELD

This document relates generally to medical systems, and more particularly, but not by way of limitation, to systems, devices, machine-readable media, and methods for using healthcare-related data to improve patient monitoring, ease patient triage, and provide treatment options for implanted electrical stimulation for treatment and/or management of pain.

### BACKGROUND

Chronic pain, such as pain present most of the time for a period of six months or longer during the prior year (or other definition of chronic), is a highly pervasive complaint and consistently associated with mental, physical, and/or psychological illnesses. Chronic pain can originate with a trauma, injury or infection, or there can be an ongoing cause of pain. Chronic pain can also present in the absence of any past injury or evidence of body damage. Common chronic pain can include headache, low back pain, cancer pain, arthritis pain, neurogenic pain (pain resulting from damage to the peripheral nerves or to the central nervous system), somatic pain, psychogenic pain (pain not due to past disease or injury or any visible sign of damage inside or outside the nervous system), or other types of pain.

However, chronic pain is far more than the existence of a physical sensation or feeling of pain. Chronic pain is not just a number rating on a scale but can be a life-consuming change to every moment of every day for a pain patient. Chronic pain has a significant impact on a person's quality of life, affecting not only physical health but also emotional and social well-being. Chronic pain can affect a patient's physical limitations, cause emotional and psychological distress, increase social isolation, bring about financial strain, decrease quality of life, and much more.

Implantable devices may be configured to sense nerve activity, such as evoked compound action potentials (ECAPs). The neural sensor may be its own device or may be part of a therapy-delivery device. The delivered therapy may include electrical therapy or drug therapy, for example. By way of example, the implantable, attachable, wearable, or similar devices may be neuromodulators that are also capable of delivering neuromodulation therapy. An example may also include cardiac stimulators that monitor nerve activity.

Neurostimulation, also referred to as neuromodulation, has been proposed as a therapy for a number of conditions, injuries, and causes of chronic pain. Examples of neurostimulation include Spinal Cord Stimulation (SCS), Deep Brain Stimulation (DBS), Peripheral Nerve Stimulation (PNS), Functional Electrical Stimulation (FES), and the like. Implantable neurostimulation systems have been applied to deliver such a therapy. An implantable neurostimulation system can include an implantable neurostimulator, also referred to as an implantable pulse generator (IPG), and one or more implantable leads, paddles, or the like each including one or more electrodes. An external trial stimulator (ETS) is an external neurostimulation device used for temporary trial periods, usually prior to receiving an IPG. The implantable neurostimulator delivers neurostimulation energy through one or more electrodes placed on or near a target site in the patient's system.

Remote patient monitoring platforms for patients with neurostimulation devices allow clinicians, device representatives, patients, and caregivers to monitor large patient populations. However, it can be difficult and time consuming to go through every patient to determine if they need help. It is desired to improve the management, triaging, and notifications provided to remotely managed patient populations.

### SUMMARY

Various embodiments of the present subject matter categorize a pain patient into one of several therapeutic categories according to parameterizing techniques for sub-perception therapy and multi-sensor paresthesia therapy, provide patient treatment changes remotely, monitor parameters, or a combination thereof.

Described implementations of the subject matter can include one or more features, alone or in combination as illustrated below by way of example.

An example (e.g., “Example 1”) of subject matter may include a system for parameterizing a closed-loop algorithm for providing sub-perception therapy, the system comprising: a stimulation output circuit configured to deliver neurostimulation; a sensing circuit configured to sense a neural signal indicative of neural responses, the neural responses each being a response to delivery of the neurostimulation; a stimulation control circuit coupled to the stimulation output circuit and the sensing circuit, the stimulation control circuit configured to control the delivery of the neurostimulation using a plurality of stimulation parameters; and one or more memory storing instructions, which when executed by one or more processors, cause the one or more processors to perform operations that: record evoked compound action potential (ECAP) features received from the sensing circuit; adjust stimulation parameters based, at least in part, on the recorded ECAP features in order to maintain consistent neural activation; record accelerometer data; generate a regression model for mapping the accelerometer data to an optimal stimulation parameter to achieve consistent nerve activation, the mapping enabling closed-loop control of the sub-perception therapy; apply the regression model and a sub-perception dose level to a plurality of sub-perception therapy programs; and deploy the regression model to a patient via the stimulation output circuit to deliver the neurostimulation.

In Example 2, the subject matter of Example 1 may optionally be configured such that the instructions perform the operations that: identify a target range for the ECAP features, the target range including an upper value of an ECAP range and a lower value of the ECAP range; and determine the consistent nerve activation by maintaining values of the ECAP features within the target range.

In Example 3, the subject matter of any one or more of Examples 1-2 may optionally be configured such that the regression model is selected from at least one of a linear regression model, support vector machine mode, neural network model, autoregressive model, or moving average model.

In Example 4, the subject matter of any one or more of Examples 1-3 may optionally be configured such that the instructions perform the operations that: prompt the patient to perform one or more physical movements; and receive, via a user interface, patient input accounting for environmental factors that impact sensor readings.

In Example 5, the subject matter of Example 4 may optionally be configured such that the one or more physical movements include one or more of a plurality of postural changes performed by the patient, the plurality of postural changes including at least one of lying down, standing up, bending over, twisting sidewise, performing daily life activities, and reclining.

In Example 6, the subject matter of any one or more of Examples 1-5 may optionally be configured such that the instructions perform the operations that: identify one or more settings in which to suggest alterations for delivering the neurostimulation to the patient, the neurostimulation being at least partially defined based on the accelerometer data.

In Example 7, the subject matter of any one or more of Examples 1-6 may optionally be configured such that the instructions perform the operations that: provide a monitoring platform; and receive information on a plurality of accelerometer data, the plurality of accelerometer data representing an overall patient health based on a combination of neurostimulation parameters comprising: an electrical waveform; and a selection of electrodes through which the electrical waveform is delivered.

In Example 8, the subject matter of Example 7 may optionally be configured such that the instructions perform the operations that: generate a suggestion for one or more additional combinations of the neurostimulation parameters; and provide the suggestion to the monitoring platform.

In Example 9, the subject matter of any one or more of Examples 1-8 may optionally be configured such that the plurality of accelerometer data provides supplemental data to the ECAP features to optimize stimulation in a sub-perception domain.

In Example 10, the subject matter of any one or more of Examples 1-9 may optionally be configured such that the system is further configured to analyze data for neurostimulation programming.

In Example 11, the subject matter of any one or more of Examples 1-10 may optionally be configured such that the instructions perform the operations that: initialize the closed-loop algorithm based at least in part on historical patient data; and adjust the closed-loop algorithm using patient-specific data.

In Example 12, the subject matter of any one or more of Examples 1-11 may optionally be configured such that the instructions perform the operations that: recalibrate the mapping to account for changes identified in an implantable pulse generator (IPG), the IPG configured to deliver electrical stimulation through leads and electrodes.

In Example 13, the subject matter of any one or more of Examples 1-12 may optionally be configured such that the regression model is further configured to generate a customized mapping for each patient by collecting ECAP and accelerometer data in a clinical setting.

In Example 14, the subject matter of Example 13 may optionally be configured such that the regression model collects ECAP factors during the mapping, the ECAP factors selected from at least one of an amplitude, a detection threshold, a perception level, a linearity with stimulation intensity, stimulation parameters, an electrode location, or neural activation levels.

In Example 15, the subject matter of any one or more of Examples 1-14 may optionally be configured to further include an anomaly detector configured to identify divergent outputs between an ECAP algorithm and an accelerometer algorithm.

Example 16 includes subject matter (such as a method, means for performing acts, machine readable medium including instructions that when performed by a machine cause the machine to perform acts, or an apparatus to perform). The subject matter may include parameterizing a closed-loop algorithm for providing sub-perception therapy, the method comprising: delivering neurostimulation; sensing a neural signal indicative of neural responses, the neural responses each being a response to the delivering of the neurostimulation; controlling the delivering of the neurostimulation using a plurality of stimulation parameters; recording evoked compound action potential (ECAP) features received from a sensing circuit; adjusting stimulation parameters based, at least in part, on the recorded ECAP features in order to maintain consistent neural activation; recording accelerometer data; generating a regression model for mapping the accelerometer data to an optimal stimulation parameter to achieve consistent nerve activation, the mapping enabling closed-loop control of the sub-perception therapy; applying the regression model and a sub-perception dose level to a plurality of sub-perception therapy programs; and deploying the regression model to a patient via a stimulation output circuit to deliver the neurostimulation.

In Example 17, the subject matter of Example 16 may optionally be configured to further include identifying a target range for the ECAP features, the target range including an upper value of an ECAP range and a lower value of the ECAP range; and determining the consistent nerve activation by maintaining values of the ECAP features within the target range.

In Example 18, the subject matter of any one or more of Examples 16-17 may optionally be configured to further include prompting the patient to perform one or more physical movements; and receiving, via a user interface, patient input accounting for environmental factors that impact sensor readings.

In Example 19, the subject matter of Example 18 may optionally be configured such that the one or more physical movements include one or more of a plurality of postural changes performed by the patient, the plurality of postural changes including at least one of lying down, standing up, bending over, twisting sidewise, performing daily life activities, and reclining.

In Example 20, the subject matter of any one or more of Examples 16-19 may optionally be configured to further include identifying one or more settings in which to suggest alterations for delivering the neurostimulation to the patient, the neurostimulation being at least partially defined based on the accelerometer data.

In Example 21, the subject matter of any one or more of Examples 16-20 may optionally be configured to further include identifying one or more settings in which to suggest alterations for delivering the neurostimulation to the patient, the neurostimulation being at least partially defined based on the accelerometer data.

In Example 22, the subject matter of any one or more of Examples 16-21 may optionally be configured to further include providing a monitoring platform; and receiving information on a plurality of accelerometer data, the plurality of accelerometer data representing an overall patient health based on a combination of neurostimulation parameters comprising: an electrical waveform; and a selection of electrodes through which the electrical waveform is delivered.

In Example 23, the subject matter of Example 22 may optionally be configured to further include generating a suggestion for one or more additional combinations of the neurostimulation parameters; and provide the suggestion to the monitoring platform.

In Example 24, the subject matter of any one or more of Examples 16-23 may optionally be configured such that the plurality of accelerometer data provides supplemental data to the ECAP features to optimize stimulation in a sub-perception domain, and further comprising: analyzing data for neurostimulation programming.

In Example 25, the subject matter of any one or more of Examples 16-24 may optionally be configured to further include initializing the closed-loop algorithm based at least in part on historical patient data; and adjusting the closed-loop algorithm using patient-specific data.

In Example 26, the subject matter of any one or more of Examples 16-25 may optionally be configured to further include recalibrating the mapping to account for changes identified in an implantable pulse generator (IPG), the IPG configured to deliver electrical stimulation through leads and electrodes.

In Example 27, the subject matter of any one or more of Examples 16-26 may optionally be configured such that the regression model is further configured to generate a customized mapping for each patient by collecting ECAP and accelerometer data in a clinical setting, and wherein the regression model collects ECAP factors during the mapping, the ECAP factors selected from at least one of an amplitude, a detection threshold, a perception level, a linearity with stimulation intensity, stimulation parameters, an electrode location, or neural activation levels.

An example (e.g., “Example 28”) of subject matter may include a machine-storage medium embodying instructions that, when executed by a machine, cause the machine to perform operations comprising: delivering neurostimulation; sensing a neural signal indicative of neural responses, the neural responses each being a response to the delivering of the neurostimulation; controlling the delivering of the neurostimulation using a plurality of stimulation parameters; recording evoked compound action potential (ECAP) features received from a sensing circuit; adjusting stimulation parameters based, at least in part, on the recorded ECAP features in order to maintain consistent neural activation; recording accelerometer data; generating a regression model for mapping the accelerometer data to an optimal stimulation parameter to achieve consistent nerve activation, the mapping enabling closed-loop control of a sub-perception therapy; applying the regression model and a sub-perception dose level to a plurality of sub-perception therapy programs; and deploying the regression model to a patient via a stimulation output circuit to deliver the neurostimulation.

In Example 29, the subject matter of Example 28 may optionally be configured to further include identifying a target range for the ECAP features, the target range including an upper value of an ECAP range and a lower value of the ECAP range; and determining the consistent nerve activation by maintaining values of the ECAP features within the target range.

In Example 30, the subject matter of any one or more of Examples 28-29 may optionally be configured to further include prompting the patient to perform one or more physical movements; and receiving, via a user interface, patient input accounting for environmental factors that impact sensor readings.

In Example 31, the subject matter of Example 30 may optionally be configured such that the one or more physical movements include one or more of a plurality of postural changes performed by the patient, the plurality of postural changes including at least one of lying down, standing up, bending over, twisting sidewise, performing daily life activities, and reclining.

In Example 32, the subject matter of any one or more of Examples 28-31 may optionally be configured to further include identifying one or more settings in which to suggest alterations for delivering the neurostimulation to the patient, the neurostimulation being at least partially defined based on the accelerometer data.

In Example 33, the subject matter of any one or more of Examples 28-32 may optionally be configured to further include providing a monitoring platform; and receiving information on a plurality of accelerometer data, the plurality of accelerometer data representing an overall patient health based on a combination of neurostimulation parameters comprising: an electrical waveform; and a selection of electrodes through which the electrical waveform is delivered.

In Example 34, the subject matter of Example 33 may optionally be configured to further include generating a suggestion for one or more additional combinations of the neurostimulation parameters; and provide the suggestion to the monitoring platform.

An example (e.g., “Example 35”) of subject matter may include a system for parameterizing a model for providing sub-perception therapy, the system comprising: one or more hardware processors of a machine; and at least one memory storing instructions that, when executed by the one or more hardware processors, cause the system to perform operations comprising: recording evoked compound action potential (ECAP) features received from a sensing circuit; adjusting stimulation parameters based, at least in part, on the recorded ECAP features in order to maintain consistent neural activation; recording accelerometer data; generating a regression model for mapping the accelerometer data to an optimal stimulation parameter to achieve consistent nerve activation, the mapping enabling closed-loop control of the sub-perception therapy; applying the regression model and a sub-perception dose level to a plurality of sub-perception therapy programs; and deploying the regression model to a patient via a stimulation output circuit to deliver neurostimulation.

Example 36 is at least one machine-readable media including instructions that, when executed by processing circuitry, cause the processing circuitry to perform operations to implement any one or more of Examples 1-35.

Example 37 is an apparatus comprising means to implement any one or more of Examples 1-35.

Example 38 is a system to implement any one or more of Examples 1-35.

Example 39 is a method to implement any one or more of Examples 1-35.

This Summary is an overview of some of the teachings of the present application and not intended to be an exclusive or exhaustive treatment of the present subject matter. Further details about the present subject matter are found in the detailed description, figures, and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE SEVERAL VIEWS OF THE DRAWINGS

This document relates generally to medical systems, and more particularly, but not by way of limitation, to systems, devices, machine-readable media, and methods for using healthcare-related data to improve patient monitoring, ease patient triage, provide treatment options for implanted electrical stimulation for treatment and/or management of pain, and parameterizing techniques for sub-perception therapy and multi-sensor paresthesia therapy. Various embodiments are illustrated by way of example in the figures of the accompanying drawings. Such embodiments are demonstrative and not intended to be exhaustive or exclusive embodiments of the present subject matter.

FIG. 1 illustrates an embodiment of a monitoring system that can include device(s), external system(s), or other healthcare related data source(s) configured for use to collect healthcare-related data for characterization of parameterizing techniques for sub-perception therapy and multi-sensor paresthesia therapy, in accordance with one embodiment.

FIG. 2 illustrates an example embodiment of data in a feature plot, in accordance with one embodiment.

FIG. 3 illustrates a series of accelerometer graphs, in accordance with one embodiment.

FIG. 4 illustrates a plot that defines a sub-perception scaling factor, in accordance with one embodiment.

FIG. 5 illustrates, by way of example and not limitation, a series of accelerometer graphs, in accordance with one embodiment.

FIG. 6, by way of example and not limitation, a stimulation amplitude graph, in accordance with one embodiment.

FIG. 7 illustrates, by way of example and not limitation, a process flow chart, in accordance with one embodiment.

FIG. 8 illustrates, by way of example and not limitation, an embodiment of a stimulation device and a lead system including ECAP factors and sensing control, in accordance with one embodiment.

FIG. 9 illustrates, by way of example, an embodiment of data interactions among a data analysis computing system and clinician and patient interaction computing devices, for operation or monitoring of a neurostimulation device based on text input analysis, in accordance with one embodiment.

FIG. 10 illustrates, by way of example, a block diagram of an embodiment of a system (e.g., a computing system) implementing neurostimulation programming circuitry to cause programming of an implantable electrical neurostimulation device, in accordance with one embodiment.

FIG. 11 illustrates, by way of example, a block diagram of an embodiment of a system (e.g., a computing system) for performing patient data analysis in connection with programming operations, in accordance with one embodiment.

FIG. 12 illustrates, by way of example, an embodiment of a neurostimulation system, in accordance with one embodiment.

FIG. 13 illustrates, by way of example and not limitation, a patient system, and examples of devices that may make up components of the patient system, in accordance with one embodiment.

FIG. 14 illustrates, by way of example, an embodiment of a programming system and data analysis system for use with a neurostimulation system, such as the implantable neuromodulation system of FIG. 17, in accordance with one embodiment.

FIG. 15 illustrates, by way of example, an embodiment of data interactions among a data analysis computing system and clinician and patient interaction computing devices, for operation or monitoring of a neurostimulation device based on text input analysis, in accordance with one embodiment.

FIG. 16 illustrates, by way of example, an embodiment of a data processing flow for affecting the neurostimulation treatment of a human patient, based on text and device data processing, in accordance with one embodiment.

FIG. 17 illustrates, by way of example and not limitation, the neuromodulation system of FIG. 12 implemented in a spinal cord stimulation system or a deep brain stimulation system, in accordance with one embodiment.

FIG. 18 is a block diagram illustrating a machine in the example form of a computer system, within which a set or sequence of instructions can be executed to cause the machine to perform any one of the methodologies discussed herein, in accordance with one embodiment.

### DETAILED DESCRIPTION

The following detailed description of the present subject matter refers to the accompanying drawings which show, by way of illustration, specific aspects, and embodiments in which the present subject matter can be practiced. These embodiments are described in sufficient detail to enable those skilled in the art to practice the present subject matter. Other embodiments can be utilized, and structural, logical, and electrical changes can be made without departing from the scope of the present subject matter. References to “an,” “one,” or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

### Overview

Example embodiments of systems, methods, machine-readable media, software, and artificial intelligence provide for improved approaches for augmenting therapy, such as deep brain stimulation and/or spinal cord stimulation, based on recording ECAP signals and/or additional indicators for adapting stimulation therapies for patients receiving sub-perception therapy, multi-sensor paresthesia therapy, and the like. Example embodiments of the present disclosure provide for systems to reconcile the use of one or more ECAP signals for parameterizing closed-loop algorithms for sub-perception therapy and multi-sensor paresthesia therapy without visualizing, recording, and/or having access to the ECAP signals at or around the time period of implementing changes to a therapy (e.g., therapeutic program).

Existing approaches of chronic pain treatment include conventional in-person visits to a clinic, telehealth appointments, or other scheduled doctor visits, which require person-to-person pain assessments in a clinical setting. Often, a patient will end up needing to provide detailed feedback to a clinician before a treatment issue can be identified and changes can be implemented to a neurostimulation treatment; this can take weeks or even months from onset to triage to treatment implementation. Conventional neurostimulation systems are insensitive to the possibilities that although chronic pain may limit patient functional capacity and cause mobility issues, an improvement in pain sensation (e.g., a lower pain rating) may not always be accompanied by or synchronized with progress in the patient's functional capacities, mobility status, and overall quality of life. For example, a chronic pain patient can report a sizable reduction in pain score yet remains to be bedridden without gaining improvement in their physical or functional capacities. In another example, a chronic pain patient may not report substantial pain reduction, even though they have started walking, sleeping normally, engaging in more activities, changing activities of living, or the like. Such conventional systems fail to provide pain patient support, monitoring, and triaging in everyday life. In addition, existing chronic pain monitoring mechanisms fail to consider or incorporate the vast amount of mental, physical, and functional pain data available to the patient throughout their daily living.

Further existing technologies for sensing nerve activity, such as evoked compound action potentials (ECAPs) may be sensed using implanted electrodes. However, implanted electrodes may be adversely affected by changes over time, such as impedance variation, lead migration, scar tissue, similar physical or physiological issues, or a combination thereof. In existing technologies, based on current ECAP recording devices, ECAP signals have limitations that can only be recorded (e.g., provide data for visualization by a user, further analysis by a computer or other computing device, such as a microcontroller) at perception levels of stimulation or above perception levels of stimulation in a patient. When a patient receives paresthesia-based therapy (e.g., perception-based, above perception level, feeling therapy, etc.), the ECAP signals (e.g., levels) typically can be recorded with electrophysiological measurements and identified by computer code-based analysis, or, for example, by visual inspection by a human operator or machine learning model. However, due to the lack of ECAP signal recordings, when sub-perception (e.g., fast-acting sub-perception therapy (FAST), no-feel therapy programs, etc.) therapy is applied to a patient, at any given time period and/or during any given patient action, the ECAPs may not be recorded with electrophysiological measurements or otherwise be available for visual inspection at or around a moment of changes being made to the therapy.

Prior approaches for a neurostimulation system depend on its post-manufacturing programmability to a great extent. One limiting factor for applications of neurostimulation therapies is that, even if a number of advanced programs can be applied by a neurostimulation device, there is often a delay for implementing new or improved neurostimulation treatments. Such a delay can be due to the infrequency of care provided by a clinician or other medical professional who oversees the treatment, and a lack of clear information regarding the results of the treatment (e.g., whether or not sub-perception therapies are providing relief to the patient). Various approaches for neurostimulation programming and customization have attempted more dynamic forms of open-loop and closed-loop programing, to allow new neurostimulation parameters or programs to be introduced, deployed, tested, and adjusted by a clinician, the subject patient, a software program, model, or the like. Although some neurostimulation devices provide the capability to enable a patient to switch between programs or change the level of a certain stimulation effect, it is often unclear whether such changes (or, which changes) are beneficial to a patient and result in improvement to the patient's medical condition.

Example embodiments of the present disclosure improve upon existing models and overcome such current technical challenges by providing sub-perception therapy that includes a substantial and growing portion of stimulation therapies for chronic pain patients and other users based on evoked responses. Evoked responses may not be available below a perception threshold in many patients, and especially not at the fractional stimulation amplitudes at which sub-perception therapy is typically programmed (e.g., 20-50% of perception threshold). Other sensors, including an on-board accelerometer, are available in both domains (e.g., sub-perception and supra-perception), and so a mapping between the two domains may enable closed-loop therapy in sub-perception based on evoked responses. The mapping between ECAP and accelerometer data will be generated clinically for each patient by collecting data and constructing regression models, in other examples, a mapping between stimulation parameters controlled by ECAP levels and accelerometer data will be generated. In additional examples, a second sensor (or one or more additional sensors) for performing mapping(s) to closed-loop outputs can enhance general algorithm performance and make robust to noise, uncertain environments, and assist in anomaly detection. For example, the closed-loop functionality can include the models and/or algorithms used by a neurostimulator to update a therapy parameter (e.g., amplitude) based on sensor data (e.g., ECAP signal, accelerometer, etc.).

According to example embodiments, sub-perception and perception therapies are effective (e.g., have a therapeutic effect on a patient, provide relief to a patient, deliver pain relief to a patient, and the like). While not all perception and/or sub-perception levels record ECAPs, therapeutic properties, such as activation of neural activity on one or more patient nerves, can still be provided to a patient receiving the therapy. In some examples, once evoked compound action potentials (ECAPs) recordings are visualized, the ECAP recordings can start to grow in size; for example, the ECAP recordings can grow linearly with the stimulation intensity that is inputted in a patient's therapy. However, below a certain stimulation intensity, ECAP levels may not be recorded (or otherwise seen or visualized), while nerve fiber activation is still occurring, despite being below a sensitivity level (or noise level of a recording system). For example, the availability of ECAP signals during perception versus sub-perception therapies may vary, and while therapeutic effects have been established at both levels, ECAP levels exhibit a detection threshold below which they cannot be observed, for technological rather than physiological reasons. Their amplitude grows approximately linearly with stimulation intensity once detectable; for example, the system may empirically observe ECAPs slightly below patients' reported perception levels.

However, stimulation therapies often target amplitudes of 20-40% of perception, generally below the ECAP detection threshold. Example embodiments use accelerometer supplementation data to enable ECAP-based control in this sub-perception domain. For example, the ECAP recording(s) can represent a large number (or larger numbers) of data points as intensity is increased (as a patient gets higher in intensity of stimulation) of the individual fibers being activated (in some examples, all at one time). In some examples, ECAP levels can be recorded at or below a level that is reported by a patient (e.g., a level of feeling or interpreted feeling by a patient). For example, ECAP recordings can be expected anywhere between, for example, 75% (percent) and 80% of a perception threshold of a therapy (e.g., sub-perception therapy, multi-sensor paresthesia therapy, etc.) being programmed for a patient. For example, the stimulation amplitude at approximately 20% of perception threshold can be recorded by an ECAP recorder. In some example embodiments, the therapeutic effect has been well established and there are underlying neural mechanisms that are being engaged but the neural mechanisms can occur at a different level.

According to example embodiments of the present disclosure, when ECAPs are being measured, the ECAPs are being measured by sensors that exist or are available currently at this state of technology. According to example embodiments of the present disclosure, the device will have circuitry that is capable of capturing electrophysiological signals from, for example, a non-stimulating contact of one or more leads (or paddles) so the non-stimulating contacts on one or all contacts on the lead will have different paths (e.g., two or more paths) back to a stimulator that is configured to connect the leads. For example, if there were two different paths, the source of current, which would provide the therapy and then another path that would conform to an analog to digital converter and amplifier circuit that could condition the signal for it to be measured and processed by a stimulator (e.g., stimulator hardware, software, or a combination thereof). Example embodiments of the present disclosure can apply to a variety of versions of a stimulation system, device, and/or method; for example, an implementation of stimulator using leads or an implementation of a stimulator using paddle(s), which may interface with a stimulator in the same, similar, or different manner (e.g., circuitry, connections, etc.).

Examples of the sub-perception space can then add additional circuitry that is configured or programmed to perform a mapping. According to example embodiments presented herein, the examples provide for spinal cord stimulation, and depending on the intensity of stimulation, a patient (and the applicable programming) can observe an evoked response. Embedded in circuitry of the stimulation device is an accelerometer that is configured (e.g., capable) to measure orientation(s), movement(s), postural changes, or the like. For example, a three-axis accelerometer can be configured to measure patient movements in the x-axis, y-axis, z-axis, and combination thereof. Based, at least in part, on the three-axis accelerometer recordings, example embodiments of the present disclosure provide for a system, method, machine-readable media, and the like to start at a higher intensity level where embodiments provide for visualizing data related to both the ECAP recordings and accelerometer recordings. Using a relationship between the accelerometer recordings and the ECAP recordings, or the accelerometer recordings and stimulation parameters (if a closed-loop control of the ECAP was being performed), examples can link (e.g., associate, correlate, etc.) the accelerometer and the stimulation amplitude recordings directly.

Based on the relationship established, a map that relates to either orientation or position (or both) is the patient in one or all three axes. For example, the mapping can provide data related to what ECAP range does that correspond to or what stimulation amplitude in order to keep the ECAP range constant. The mapping can provide information related to how each stimulation parameter (or a combination of stimulation parameters) corresponds to sub-perception therapy and multi-sensor paresthesia therapy, according to example embodiments. For example, in order to get to the sub-perception therapy level, example embodiments could apply a scaling factor that would show the desired (e.g., wanted) level of intensity for the particular therapy to be applied to a patient. In some examples, a scaling factor can be applied to multiply to try to use accelerometer recordings to predict stimulation amplitude. For example, stimulation amplitude can be predicted in higher-intensity spaces where the system or method can track the ECAP to ensure it is constant. Then, based on the consistent ECAP recording, the ECAP level is multiplied to predict an amplitude by a sub-perception scaling factor. For example, a sub-perception scaling factor could be 20% or 10% or another number or percentage (e.g., factor) that can take the scaled dose down to a dose level wanted or desired by a user (e.g., patient, doctor, clinician, programming model, etc.) in a sub-perception range.

According to examples of the present disclosure, the mappings can be created (e.g., generated) according to an algorithm or approach that is established (e.g., constructed, prepared, etc.) in a clinic, home, or other location with a patient. For example, a representative or clinician can configure one or more intended therapies (e.g., above perception or below perception) such as stimulation programs according to clinic settings. In some examples, a clinician will begin at (e.g., start for configuration purposes) an above-perception therapeutic level where the ECAPs can be measured (e.g., recorded, viewed, visualized, etc.). In such examples, the clinician can ensure the accelerometer is working and available. For example, the example would record both the ECAP recordings and/or the accelerometer readings, while trying to hold (e.g., maintain constant) the ECAPs constant with a control algorithm that adjusts one or more of several stimulation parameters.

According to examples, a configured model can be generated as a generic (e.g., general, exemplary, etc.) model to be used as a starting point (or initial point) used across all or some patients on a variety of bases, such as on a per-pain level basis, per-condition basis, per-implementation basis, or the like. For example, different patients may have an IPG implanted in different orientations, where different patients move and/or react to stimulation or other properties in unique manners, which can be used to determine or help identify how the mapping will occur or be visualized. For example, the system can model on a per-patient basis, per-injury basis, per-symptom basis, or the like. According to some example embodiments, a patient with a stimulation device for receiving sub-perception therapy and/or multi-sensor paresthesia therapy that is enabled to measure ECAPs or in which ECAPs can otherwise be measured (e.g., an accelerometer and stimulation capabilities) can receive parameterized closed-loop models to optimize (e.g., improve, adjust, enhance) therapeutic outcomes.

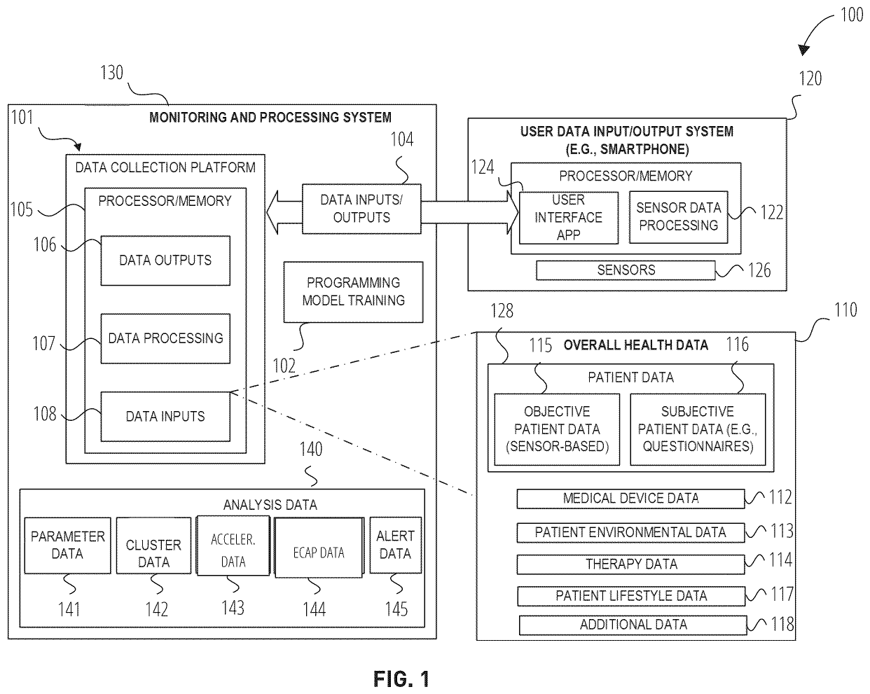

FIG. 1 illustrates a block diagram 100 of a monitoring and processing system 130 that can include medical device(s), external system(s), or other healthcare related data source(s) configured for use to collect healthcare-related data for characterization of parameterizing techniques for sub-perception therapy and multi-sensor paresthesia therapy. By way of example, an embodiment of a monitoring and processing system 130 configured to collect and analyze healthcare-related data in connection with the use of neurostimulation programs and closed-loop neurostimulation programming.

The illustrated monitoring and processing system 130 is configured to include at least one data collection platform 101, which operates to collect and provide data inputs/outputs 104. The data collection platform 101 is configured to process (e.g., filter, extract, transform) the input data to produce analysis data 140, which then can be used for one or more iterations of programming model training 102. The programming model training 102 can be closed-loop programming model, or open-loop system where the action from a controller is independent of the process output (which is the system variable that is being controlled), or other programming models that supply feedback to adjust operation of the system. The data collection platform 101 can also generate output data in the form of recommendations, suggestions, and other information which is relevant to the collection of input data.

The monitoring and processing system 130 may be implemented at one or more server(s) or other systems remotely located from the patient. The monitoring and processing system 130 may use various network protocols to communicate and transfer data through one or more networks which may include the Internet. The monitoring and processing system 130 and data collection platform 101 can include at least one processor configured to execute instructions stored in memory (e.g., depicted as processor(s)/memory 105) to generate or evaluate data outputs 106, to obtain or evaluate data inputs 108, and to perform data processing 107 on both inputs and outputs and accompanying training data.

The data inputs 108 can be processed to generate the analysis data 140, which represents a transformed or refined version of data based on actual patient usage, patient feedback (e.g., feedback that indicates what settings have been effective or have not been effective), patient parameter inputs, and the like. The analysis data 140 can include additional data processing including, for example, parameter data 141, cluster solution data 142, accelerometer data 143, ECAP data 144, and alert data 145.

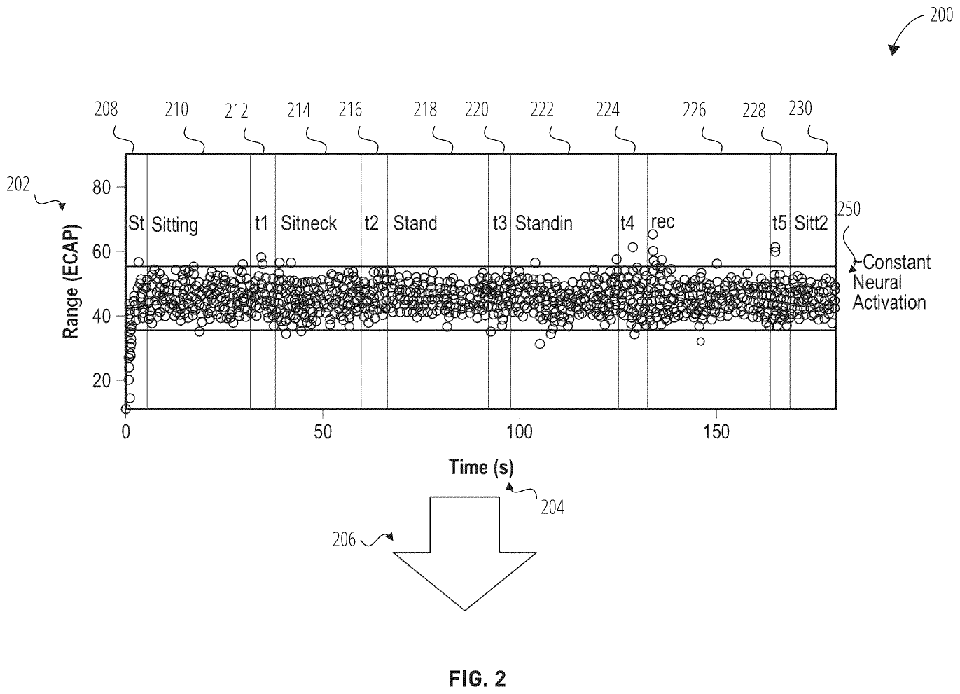

The data inputs 108 may include information obtained from the patient directly and may include various types of overall health data 110 associated with the patient or the treatment. Examples of overall health data 110 may include patient data 128, medical device data 112, patient environmental data 113, therapy data 114, patient lifestyle data 117, additional data 118, or various combinations thereof. The patient data 128 may include objective data 115, such as data collected from physiological sensor(s) and subjective data 116, such as data collected from user-answered question(s) (described in detail in connection with FIG. 2). Additional examples of how objective and subjective data is collected from a patient are discussed in more examples below.

The user data input/output system 120 may be implemented at one or more devices located at or operated by the patient, such as via a smartphone, personal computer, tablet, smart home device, a remote programmer, a programming device, or another compute device or platform capable of collecting input and providing output. The user data input/output system 120 may include at least one processor configured to execute instructions stored in memory (e.g., depicted as processor(s)/memory 105) to provide or generate the data inputs and outputs 104 for receiving or collecting overall health data 110. One such example is a user interface application 124 implemented as a graphical user interface (GUI). Examples of GUIs for data inputs and outputs include smartphone applications or remote control applications, or the like. Another example is sensor data processing 122 which can be used to determine the patient physiological state (e.g., amount of activity, duration of sleep) when a particular program is in use.

The user data input/output system 120 may include or interface with sensors 126 such as physiological sensors that can detect information (e.g., activity, sleep, etc.), event(s), indicators of device usage, patient compliance with data collection and/or therapy, or various combinations thereof. The user data input/output system 120 may directly or indirectly capture measurements from the sensors 126 or from associated external systems. Examples of external system(s) include remote controls, programmers, phones, tablets, smart watches, personal computers, and the like. The external system may be configured to provide the medical device data 112, the patient environmental data 113, the therapy data 114, patient lifestyle data 117, and/or additional data 118 from an associated device.

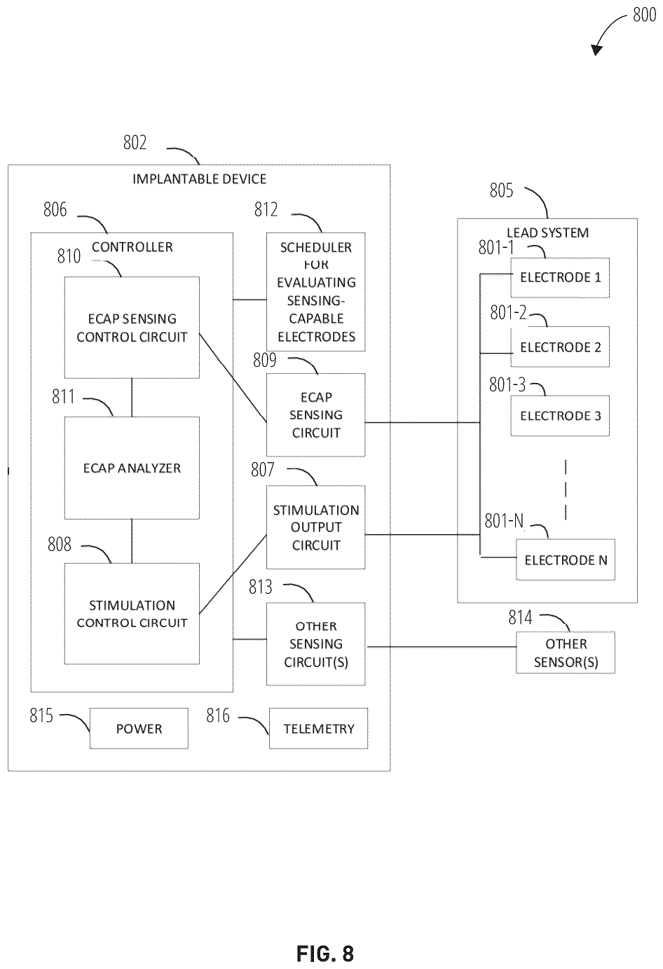

The programming model training 102 operates to update a programming model by training, re-training, or updating a model (e.g., an artificial intelligence model, such as a neural network) based on the produce analysis data 140. One or multiple instances of a model may be trained to generate programs and program parameters, including with one or more patient-specific models. The closed loop programming model training 102 may consider other aspects of patient-specific or population data such as the overall health data 110, sensor data, and rules and information from a variety of data sources. More details on an example method are described and depicted in connection with FIG. 8.

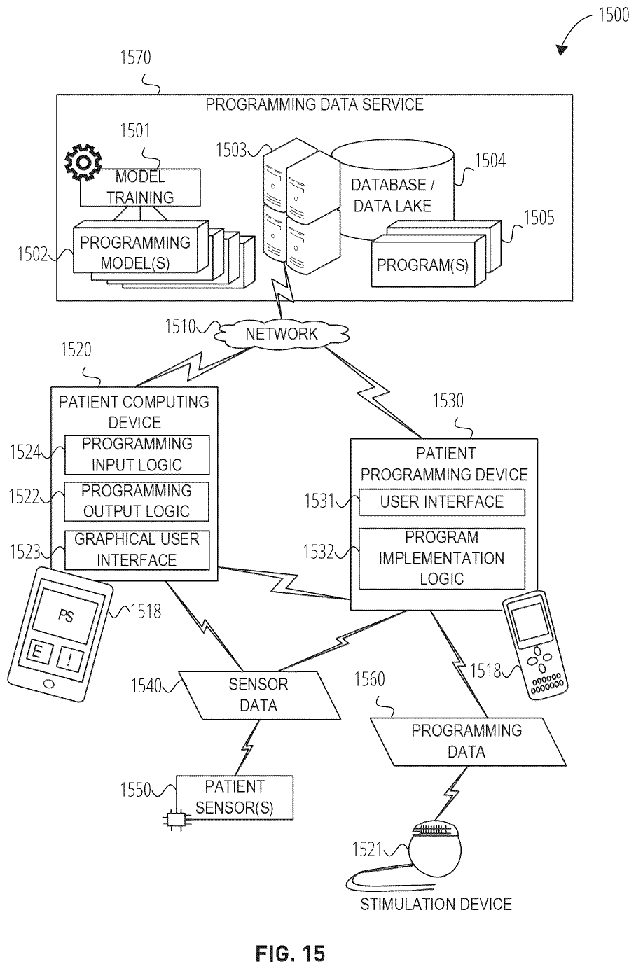

In some examples, the data collection platform 101 and monitoring and processing system 130 may directly interface with one or more medical device(s), external system(s), or other healthcare-related data source(s) to collect the overall health data 110. One or more of the medical devices, external system(s), and/or other patient-related data source(s) may include technology used by the monitoring and processing system 130 to collect and/or record data, and thus may form part of the data collection platform 101. Examples of devices or medical devices include implantable, wearable, and/or operatively interconnected devices. Implantable devices may be configured to sense nerve activity such as evoked compound action potentials (ECAPs), which can identify and record nerve conduction, feeling, activity, and other patient monitors. The implantable device, such as stimulation device 1502 as described and depicted in connection with FIG. 15, may be configured to sense nerve activity (e.g., ECAPs) using the electrodes, and may be further configured to evaluate sensing-capable electrodes as discussed within this document.

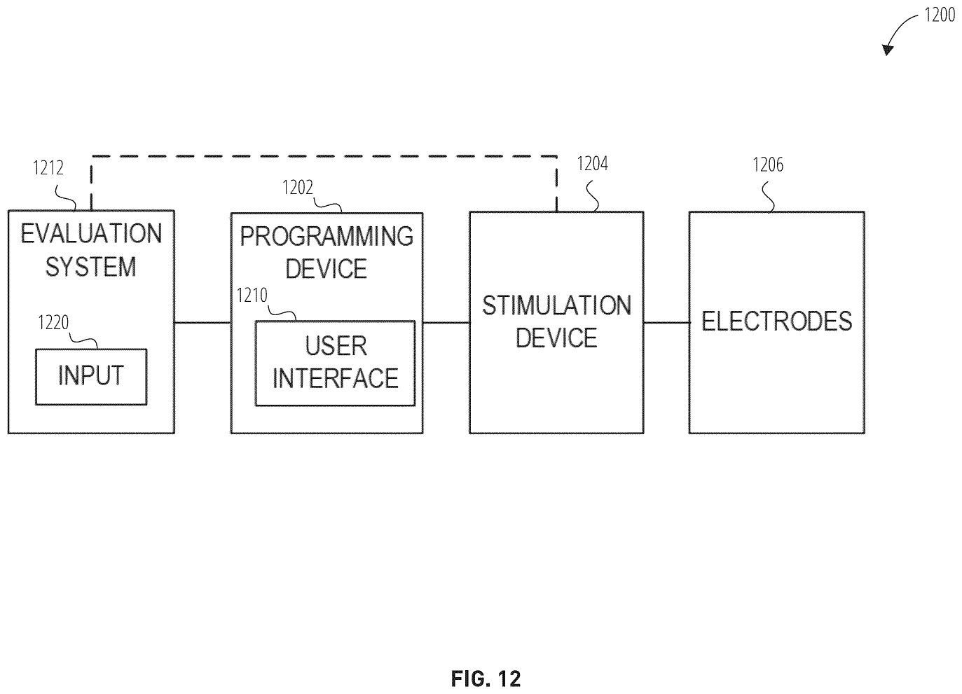

The implantable device may be also configured to deliver an electrical therapy such as a cardiac rhythm management therapy, a neuromodulation therapy, sub-perception therapy, multi-sensor paresthesia therapy, similar therapies, or combinations thereof. Therefore, by way of example and not limitation, the implantable device may be a neuromodulator configured to deliver neuromodulation energy, such as in the form of electrical pulses, to the one or more neural targets (between upper values and lower values of an ECAP range) though electrodes, such as electrodes 1206 as described and depicted in connection with FIG. 12. The delivery of the neuromodulation may be controlled using a plurality of modulation parameters that may specify the electrical waveform (e.g., pulses, pulse patterns, other waveform shapes, etc.) and a selection of electrodes through which the electrical waveform is delivered. The existing waveforms stored in the external storage device can be definable at least in part by one or more parameters including, but not limited to the following: amplitude, pulse width, frequency, duration(s), electrode configurations, total charge injected per unit time, cycling (e.g., on/off time), waveform shapes, spatial locations of waveform shapes, pulse shapes, number of phases, phase order, interphase time, charge balance, and ramping.

In various example embodiments, at least some parameters of a plurality of modulation parameters are programmable by a user, such as a physician, caregiver, device representative, patient, hardware system, software system, or combination thereof. The programming device can provide the user with accessibility to the user-programmable parameters, such as for parameters used in parameterizing techniques for sub-perception therapy and multi-sensor paresthesia therapy.

Example embodiments of the medical device may be configured to collect only data, to deliver only therapy, or to both collect data and deliver therapy. The medical device may be configured to collect and provide medical device data such as device model, configuration, settings, and the like. Thus, the medical device may provide the patient data 128, the medical device data 112, the patient environmental data 113, therapy data 114, patient lifestyle data 117, and/or additional data 118, particularly in specialized neurostimulation programming environments.

Other healthcare-related data source(s) may include patient data 128 received via a provider's server that stores patient health records. For example, patients may use a patient portal to access their health records such as test results, doctor notes, prescriptions, and the like. Other healthcare-related data sources may include apps on a patient's smartphone or other computing device, or the data on a server accessed by those applications. In another example, an application on a phone or patient's device may include or may be configured to access environmental data such as weather data and air quality information or location elevation data such as may be determined using cellular networks and/or a global positioning system (GPS). Weather data may include, but is not limited to, barometric pressure, temperature, sunny or cloud cover, wind speed, and the like. By way of example and not limitation, this type of data may include heart rate, blood pressure, weight, and the like collected by the patient in their home. Environmental factors can be accounted for using patient input or other environmental data.

In additional examples, this type of data may include ECAP features, such as amplitude, detection threshold, perception level, linearity, stimulation parameters, electrode/lead/paddle locations, neural activation, or the like. The amplitude or size of the recorded ECAP signal provides information about the number of nerve fibers activated by the electrical stimulation. In additional examples, the temporal profile of the recorded ECAP signal provides information about the type or size of nerve fibers activated by the electrical stimulation. Larger ECAP amplitudes indicate more nerve fiber recruitment. ECAPs exhibit a detection threshold below which they cannot be observed or recorded, even if nerve activation is occurring. This is a technological limitation, not a physiological limitation. For example, in some examples, ECAPs may only be recordable at perception levels and above in patients. In additional examples, no or few ECAP signals can be detected during sub-perception stimulation. Once above the detection threshold, ECAP amplitude grows approximately linearly with increasing stimulation intensity. Factors like stimulation amplitude, pulse width, frequency affect the ECAP response and must be optimized to target fibers. The recording electrode location impacts the ECAP signal and must be carefully placed in order to detect responses.

While not always recordable, ECAPs provide objective information about the level of neural activation achieved by the stimulation (e.g., relief provided to the patient). ECAPs serve as feedback biomarkers for closed-loop systems that automatically maintain optimal stimulation levels and nerve activation. For example, ECAP characteristics and detection can vary between patients based on factors like electrode placement, nerve health, body morphology, patient movement, body posture, changes in movement, or the like. For example, key ECAP features that should be considered include amplitude, detection threshold, perception level, linearity with stimulation intensity, dependence on stimulation parameters, recording location, relationship to neural activation levels, and variability between patients.

The data inputs/outputs 104 may be provided with data transferred via at least one network. The data transfer may use various network protocols to communicate and transfer data through one or more networks which may but does not necessarily include the Internet and/or various wireless networks, which may include short range wireless technology such as Bluetooth. The data may be transferred directly from at least one of the external systems and/or may be transferred directly from at least one of the medical device(s). Further, the external system(s) may be configured to receive data from an associated medical device(s) and/or receive data from other healthcare-related data source(s), and then transfer the data through the network(s) to the data receiving system(s).

FIG. 2 illustrates an example embodiment of data in a feature plot 200, in accordance with one embodiment. The feature plot 200 displays an approximate constant neural activation range 250 plotted as a function of evoked compound action potential (ECAP) ranges 202 over times 204, in accordance with one example embodiment.

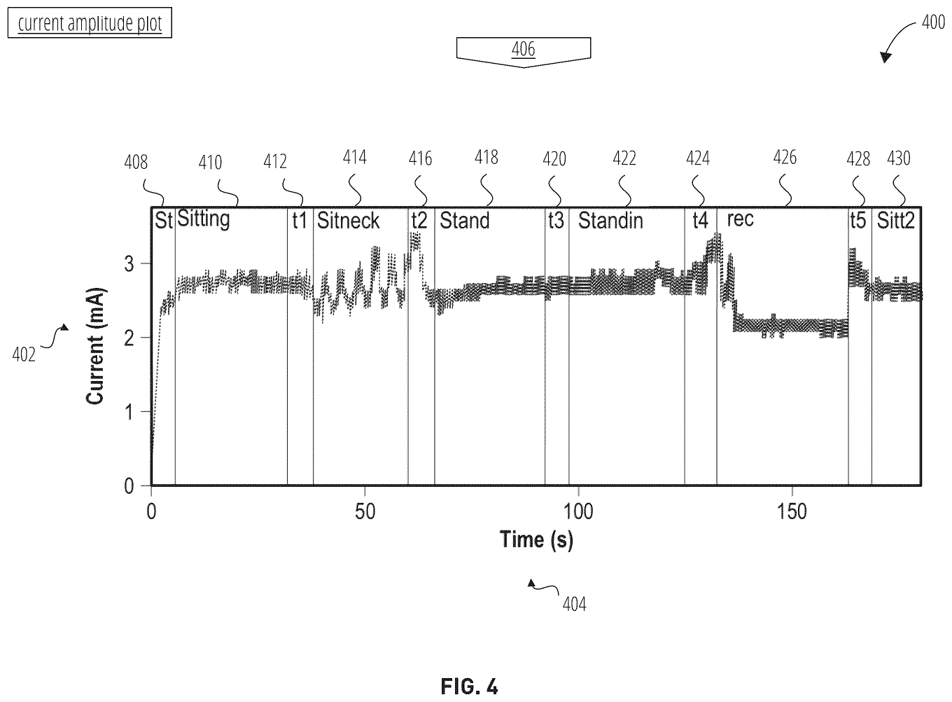

FIG. 2 illustrates a feature plot 200 representing the ECAP level, and from the ECAP level recorded or viewed, the system can extract a feature (e.g., a peak-to-peak amplitude from a region of the signal that is of interest). Based on this feature, the system can isolate a specific window of the recorded signal. In some examples, the window of the recorded signal can be based on, for example, when the signal appears (e.g., looks, measures, etc.) to be the biggest or most noticeable. At which point, the system (via a clinician programming, algorithmic model, or the like) can adjust (e.g., make adjustments to) the stimulation amplitude and/or the current (as described and depicted in current amplitude plot 400 in FIG. 4).

Returning to FIG. 2, the approximate constant neural activation range 250 in the feature plot 200 includes a series of data points (where each data point is represented by a single circle on the plot) between ECAP ranges of 0 to 80 (as displayed on the y-axis) over times 204 ranging from 0 to 200 seconds (as displayed on the x-axis), the ranges being example ranges without limitation. For example, the constant activation range can include a subset of the overall range, where the subset, for example, is between 35 and 55 in the ECAP range. The feature plot 200 demonstrates a consistent (or approximately consistent neural activation via control (e.g., closed-loop control) at a stimulation level with evoked responses present. In some examples, the evoked responses may be above a perception level, at a perception level, or below a perception level.

The hundreds of data points (which could be ten to thousands of data points depending on the feature plot example), provide data points beginning at a start 208 position over a period of approximately 200 seconds (about 3.33 minutes) of a day. However, it will be understood by those of skill in the art that data may be gathered over any period of time, whether continuous, semi-continuous, distributed, over minutes, hours, days, months, etc. In the feature plot 200, after the start 208 position, the patient moves in a variety of postural positioning movements over a period of time. For example, from the start 208 position, the patient moves to a sitting position (for a first time) 210 for about 20 seconds to time t1 212. After time t1 212, the patient moves to a sitting position (with neck reclined) 214 for about ten seconds to time t2 216. After time t2 216, the patient moves to a standing or stand-up position 218 for about 40 seconds to time t3 220. After time t3, the patient moves to a standing position 222 for approximately 30 seconds to time t4 224. After time t4 224, the patient moves to a reclining position (or in a reclined position) 226 for 40 seconds to time t5 228, at which point the patient again moves to a sitting position (for a second time) 230.

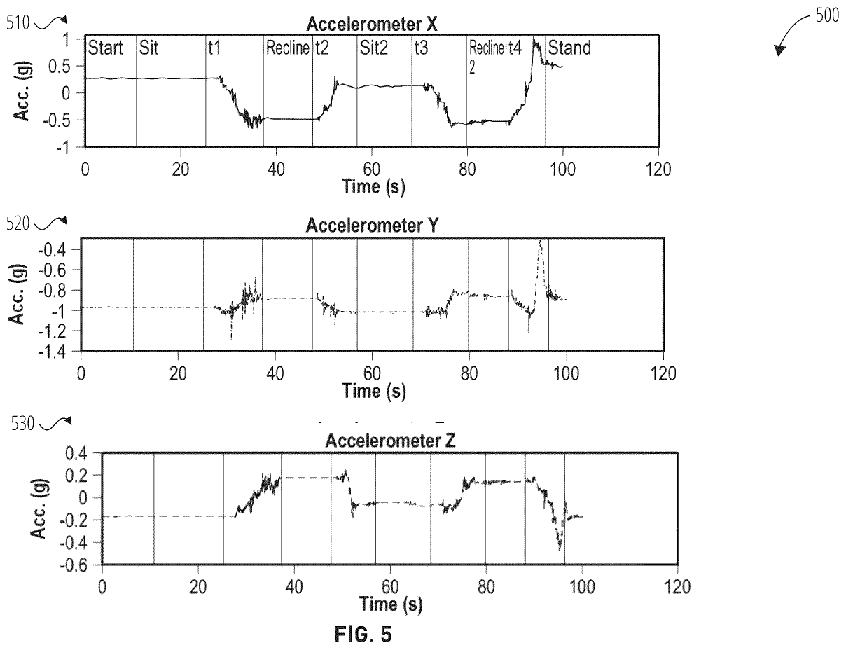

Based on the hundreds of ECAP range (ECAP) 202 data points over times 204 (approximately 0 to 200 seconds), a clustering range is generated, which represents a constant neural activation 250 range between ECAP ranges. In some examples, the aggregation of data points into dense accumulations is indicative of similarity between proximate values, which is termed ‘clustering’ in data visualization. Clustering arises when subsets of the data exhibit high congruence in their measured attributes. Identification of such clusters through graphical techniques or algorithmic methodologies can elucidate the latent structure within the data based on shared characteristics. For example, the constant neural activation 250 range can display well-defined clusters that represent modes of the distribution corresponding to categories or correlations in the underlying phenomena in ECAP ranges. For example, the number, density, shape, and separation of discernible clusters provide insight into the distribution and relationships in the ECAP range data. Cluster analysis comprises formal statistical approaches for extracting clusters from data, with popular techniques including k-means clustering, hierarchical clustering, density-based clustering, other clustering algorithms, or a combination thereof. However, not all data points may manifest clustering behavior and outliers with high dissimilarity to other observations may be present, such as in the beginning of the feature plot 200, the start 208 range may not be useful data. As the clustering provides for a natural tendency of analogous data points to congregate in data visualization, the constant neural activation 250 range allows for interpretation of patterns and categories (described and depicted in connection with FIGS. 4 and 5 below).

In some examples, the process of categorizing continuous data into discrete groupings to demonstrate consistent neural activation via closed-loop control at a stimulation level can be mapped from sensor data to stimulation parameters known as “bins” for the purposes of simplification and pattern analysis (also referred to as “data binning”). Binning can include separating a dataset into intervals, segments, ranges of values, or the like referred to as “bins.” These bins typically partition the data range into contiguous, non-overlapping intervals of equal size. Binning thereby transforms quantitative data into qualitative categories (e.g., sitting position (for a first time) 210, stand up position 218, standing position 222, etc.). This discretization of a continuous variable facilitates comprehension of the data distribution by reducing noise and highlighting trends. For example, the ECAP ranges for each bin can highlight sensor data based, at least in part or fully, on stimulation parameters. In some examples, one or more stimulation parameters can be exposed via the mapping. However, selection of appropriate bin sizes requires compromising between excessively fine partitions that introduce complexity and overly coarse groupings that obscure insights. While common bin dimensions are 5, 10 or 20 units, optimal bin width is contingent on the data distribution. As such, depending on the example, data binning can be used (as opposed to clustering or in combination therewith) as a valuable visualization approach for extricating the underlying structure of extensive datasets related to stimulation parameters through prudent aggregation into discrete bins.

According to some examples, a closed-loop machine learning model refers to a model that incorporates a feedback loop to continuously update and improve its performance based on its own outputs or predictions. This creates a closed-loop system where the model's predictions are fed back into the model as new input data. The key advantage of this mechanism is that the model can automatically and constantly learn and adapt in real-time, near real-time, or other fixed or variant time periods without needing manual updates or retraining by a human. The feedback loop enables continuous learning as new data including the model's own predictions are fed back into the model (e.g., algorithm, software, artificial intelligence, machine learning, etc.); the feedback loop can incrementally improve itself and optimize its performance. The learning process happens automatically in a dynamic, iterative manner. As conditions change or new data comes in, the model can adapt and tailor itself without human intervention.

For example, closed-loop models are well-suited for applications that require real-time decision making, control, optimization, or a combination thereof. Example use cases include robotic control systems, automated trading systems, predictive maintenance systems, recommendation engines, and more. The automated feedback loop allows the models to optimize and refine their performance continuously for these types of applications. Overall, the adaptation or learning process used by the model and/or algorithm to improve predictions over time provides an efficient and automated way for machine learning models to learn and adapt on-the-fly based on their own outputs. This real-time adaptability makes closed-loop models powerful for applications that need constant optimization and improvement in dynamic environments. The continuous feedback loop allows the models to learn by doing in an iterative, self-improving loop.

It will be understood by those having skill in the art that algorithms and models according to examples presented herein can alternatively, additionally, or otherwise employ non-adaptive or static machine learning models for implementing parameterization for sub-perception therapy according to examples of the present disclosure. However, for simplicity in explanation, closed-loop algorithms are used without limitation. For example, non-adaptive machine learning can refer to models that are trained on a fixed dataset and deployed without any feedback loop. In non-adaptive systems, models make predictions on new input data but do not use those predictions to further update themselves. Non-adaptive or static models can be trained on historical training data, validated, and then deployed statically. They do not change or adapt once deployed. The learning is open without any feedback flow. In addition, neurostimulation models where sensor feedback is not used to adjust therapy parameters can be referred to as open-loop models.

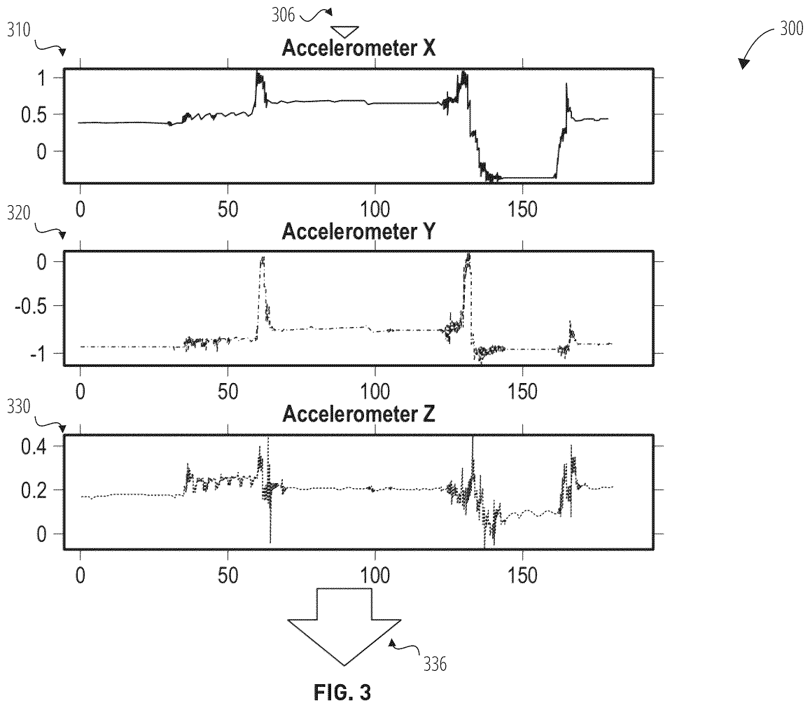

The feature plot 200 of FIG. 2 provides data that is used to create a mapping. For example, the ECAP data shown in feature plot 200 depicts consistent neural activation that is a precondition or ideal for creating (e.g., generating) the mapping between data depicted in FIGS. 3 and 4. As depicted in FIG. 2, the arrow 206 continues to the example embodiment of accelerometer data as displayed in FIG. 3.

FIG. 3 illustrates a series of accelerometer charts 300 of accelerometer graphs 310/320/330 based on the mapping provided in FIG. 2 and data received via arrow 306, in accordance with example embodiments of the present disclosure.

FIG. 3 illustrates an example of three accelerometer graphs, including Accelerometer X graph 310, Accelerometer Y graph 320, and Accelerometer Z graph 330. Each graph provides data based on accelerometer signals recorder simultaneously (or near simultaneously) that are used to create a mapping, in accordance with example embodiments of the present disclosure. For example, the mapping can include a regression model that is mapped directly from accelerometer signals (e.g., data signals, data input, etc.) to manipulated stimulation parameters. The manipulated stimulation parameters can include one or more parameters, such as amplitude, pulse width, frequency, or the like.

A variety of mathematical, algorithmic, and computational techniques exist within the domains of machine learning and classical statistics for purposes such as regression, classification, forecasting, pattern recognition, and the like that can be used to generate a mapping from the accelerometer signals to stimulation parameters, as well as for recalibrating the mapping. For example, linear regression comprises an elementary yet ubiquitous approach for modeling the relationship between a dependent variable and one or more explanatory variables through a linear functional form. In other examples, support vector machines (SVM) constitute a supervised learning methodology that constructs an optimal hyperplane for robust classification and regression tasks. Neural networks can encompass an interconnected system of nodes inspired by cerebral biology and capable of extracting complex patterns from data through sophisticated learning protocols. Autoregressive models employ regression equations wherein lagged values of a time series are exploited to predict future observations. The autoregressive designation signifies the utilization of the variable's own prior values as predictive covariates. In other examples, moving average models apply a mathematical averaging operation across a fixed window of prior time periods to smooth short-term fluctuations and elucidate long-term trends. According to the example embodiments presented herein, these (and other) paradigms offer tools for distilling insights from data across myriad applied contexts for creating a mapping directly or indirectly from accelerometer signals to raw, manipulated, or semi-manipulated stimulation parameters or other data.

In some examples, this confirmation can be confirmed by subjective measurements (e.g., asking the patient if the perception level was too intense or too weak at any point, patient input, etc.). In other examples, the confirmation can be confirmed according to objective measurements (e.g., according to physical recordings, patient states, recorded data, etc.). In additional examples, a combination of subjective and objective measurements can be used to confirm a consistent (or near-constant) perception level. Once a perception level is confirmed to be consistent, then the system can generate (e.g., create) a mapping (e.g., a regression model) that uses one or more accelerometer signals as inputs and employs, for example, a linear model to predict what would be a stimulation output in that case.

As depicted in FIG. 3, the arrow 336 continues to the example embodiment of FIG. 4, which receives the accelerometer graph data at arrow 406 and creates a current amplitude plot 400.

FIG. 4 illustrates a current amplitude plot 400 that defines a sub-perception scaling factor that attenuates a predicted amplitude from a regression model (via data from arrow 406) to an appropriate sub-perception dose to be applied to a patient's therapy, according to example embodiments.

Example embodiments of the system of the present disclosure can adjust the stimulation amplitude and/or the current 402 as expressed on the current amplitude plot 400 over time 404 (in seconds). At which point, the system can be configured to maintain the ECAP level at a consistent level or approximately constant level that provides for a more consistent neural activation level in the patient (e.g., a consistent level of therapy for the patient). In some examples, when a patient is feeling the stimulation (e.g., at or above a perception level), a consistent perception level of stimulation is provided to the patient to provide improved or ideal therapy.

For example, the current amplitude plot 400 can define a sub-perception scaling factor that attenuates the predicted amplitude from the regression model to the appropriate sub-perception dose. In some examples, the accelerometer signals captured from an accelerometer being worn by the patient continuously, semi-continuously, or variously capture data while running in the background. In additional example embodiments, such as in IPG or ETS embodiments of stimulation devices, an accelerometer can be on a chip or other circuitry, which can provide all or some number (e.g., three, five, etc.) of measurements for accelerometer sensing. One or more additional sensors (e.g., chip, circuitry, etc.) are provided for ECAP sensing, distinct from the accelerometer sensing. Once a consistent perception level is confirmed (or approximately confirmed), example embodiments are able to control the patient's perception or therapy level (e.g., specifically in the sub-perception examples) and/or neural activation level within the patient via the patient's prescribed therapy dose.

The hundreds of data points (which could be ten to thousands of data points depending on the feature plot example), provide data points beginning at a start 208 position over a period of approximately 200 seconds (about 3.33 minutes) of a day. However, it will be understood by those of skill in the art that data may be gathered over any period of time, whether continuous, semi-continuous, distributed, over minutes, hours, days, months, etc. In the current amplitude feature plot 200, after the start 408 position, the patient moves in a variety of postural positioning movements over a period of time, which is normalized according to a scaling factor. For example, from the start 408 position, the patient moves to a sitting position 410 (for a first time) for about 30 seconds to time t1 412. After time t1 412, the patient moves to a sitting position (with neck reclined) 414 for about ten seconds to time t2 416. After time t2 416, the patient moves to a standing stand-up position 418 for about 40 seconds to time t3 420. After time t3 420, the patient moves to a standing position 422 for approximately 30 seconds to time t4 424. After time t4 424, the patient moves to a reclining position (or in a reclined position) 426 for 40 seconds to time t5 428, at which point the patient again moves to a sitting position (for a second time) 430. The movements described and depicted in connection with FIGS. 2, 3, and 4 come from the same dataset with all three streams recorded. It will be understood by one skilled in the art that additional datasets, data, and/or movements can be recorded or otherwise used in identifying the same or different movements.

FIG. 5 illustrates a series of accelerometer charts 500 of accelerometer graphs 510/520/530 based on accelerometer data, which is based on a patient's movements and is correlated with and mapped to stimulation amplitude based on the derived regression model, in accordance with example embodiments of the present disclosure.

Example embodiments of the present disclosure provide for parameterization of sub-perception therapy through a process of capturing some or all of the ECAP factors (as illustrated in FIG. 2), accelerometer signals recorded simultaneously on multiple axes (as illustrated in FIG. 3), and the simulation amplitude based on closed-loop amplitude adjustment to maintain neural activation based on recorded ECAP features (as illustrated in FIG. 4). ECAP features may be one or more of: Range (e.g., max-to-min amplitude), Area Under the Curve, Curve Length, N1 or trough time, P2 or peak time, conduction velocity, or others. Once the ECAP features are recorded, the system adjusts stimulation parameters based on the recorded ECP features in order to maintain consistent neural activation. For example, consistent neural activation can entail maintaining at a consistent level one or more of the ECAP features (e.g., range, area under the curve, etc.). In other examples, the system monitors and records consistent neural activation that follows from features that are correlated with the distance between stimulating electrodes and the spinal cord, and with sub-perception closed-loop algorithms, which are generated to maintain this same level of neural activation.

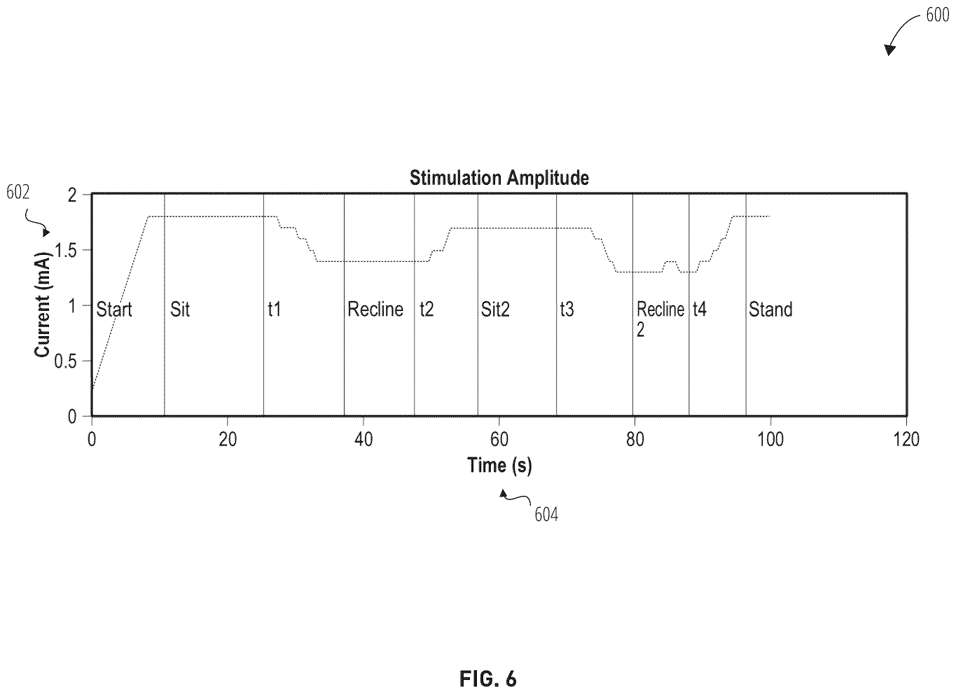

In some configurations, features, or combinations of several features, may be specifically chosen to correlate with some spinal cord physiological parameter, such as distance between the stimulating electrodes and the spinal cord. The closed-loop amplitude adjustment algorithm based on ECAP features may also have a hierarchical logic embedding based on several measured features to determine if a physiological parameter such as distance between stimulating electrodes and spinal cord were likely to have changed. Based on some or all of this initially captured data, the system configures a model between the feature plot 200, the series of accelerometer charts 300, and the current amplitude plot 400. Once the model is configured, the system scales down the configured model by a sub-perception percentage or factor, and the algorithm is executed at that level. In such example embodiments, the algorithm is only adjusting based on the accelerometer signals that are being recorded, and based on that, a new recording is predicted, and the stimulation level is scaled. For example, it may be applied to a sub-perception program at a dose equal to 50% of perception threshold in order to apply the stimulation amplitude to a patient's therapy. For example, a stimulation current amplitude plot 600 is generated to provide feedback and updated therapy to the patient, as well as provide feedback data via the closed-loop control (described and depicted in detail in connection with FIG. 6).

In additional example embodiments, an accelerometer may be slower to respond to changes, and in turn, a patient may feel changes in perception level when sub-perception therapy levels are increased (e.g., brought to or higher-then perception levels). In such examples, the system can provide a message (e.g., alert, notification, indication, etc.) to notify the patient or other user that their perception level may be changing or feeling different. In such examples, the system can use (e.g., employ, rely on, etc.) one or more ECAP signals more heavily (than the accelerometer recordings) in the model(s) to adjust the levels or parameters for sub-perception and/or multi-sensor paresthesia therapies.

FIG. 6 illustrates an example stimulation amplitude chart 600 that displays a stimulation amplitude graph based on a mapping for parameterizing closed-loop algorithms for sub-perception therapy, in accordance with one embodiment.

According to some example embodiments, methods include mapping from one or more sensor(s) directly to one or more stimulation parameters. Example methods demonstrate consistent neural activation via closed-loop control at a stimulation level with evoked responses present (likely above perception levels). Such methods can create a mapping, such as a regression model, directly from accelerometer signals to manipulated stimulation parameter(s), such as amplitude, in this case, but such parameters can include, for example, pulse width, frequency, or the like. The mapping can be generated using, for example, methods such as linear regression, SVM, neural network, autoregressive, moving average, etc. The graphs, such as the series of accelerometer charts 300 as described and depicted in connection with FIG. 3, display accelerometer signals recorded simultaneously that can be analyzed and projected to identify a sub-perception scaling factor.

After the accelerometer signals are analyzed and projected to identify a sub-perception scaling factor, a current amplitude plot can be run or generated to define a sub-perception scaling factor that attenuates the predicted amplitude from the regression model to the appropriate sub-perception input (e.g., dose of therapy). For example, a current amplitude plot can display a closed-loop amplitude adjustment to maintain neural activation in a patient during a variety of activities (e.g., movements, changes in orientation, etc.) occurring over a period of time. The amplitude adjustment can provide a stimulation amplitude to provide to a user that includes a dose parameter, such as current 602 to be set to a sub-perception level for periods of time(s) 604 based on the accelerometer data recorded from a patient.

In some examples of creating a mapping, when the system generates a mapping and/or when the system uses the generated mapping, examples can toggle (e.g., slide, move, rotate, etc.) data along a window. For example, on a sliding-scale of time (e.g., approximately one second of data), at each time marker, the system can implement or perform an update of the signal level to form (e.g., calculate) averages that can be used as input (e.g., such as input to a regression model). For example, the update can be based on an average (or other median, etc.) of each of the last X seconds of data, which can be used as a time window that the parameterizing closed-loop algorithm employs. For example, a step-size of the algorithm can be determined based on the prior (e.g., last) X seconds of data to determine how to change the stimulation output. For example, X seconds of data could be half of a second, 1 second, 5 seconds, 10 seconds, or other step-size or average step-size of data for a window (or orders thereof). The step-size, or average step-size can then be tuned and/or adjusted around a patient's movement needs profile, pain therapy desires, patient state, or the like.

In additional example systems, methods, machine-readable medium, and the like provide for visualizing data related to both the ECAP recordings and accelerometer recordings. In extreme (or rare) occurrences, a patient may be in a physical location that effects the ECAP recordings and/or the accelerometer readings. Examples affecting the ECAP recordings may include going through airport or retail security scanners, being near medical imaging equipment, the ECAP sensing circuitry encountering some fault condition that prevents proper ECAP recording, or the like. Examples affecting accelerometer data may include if the patient was under water (e.g., below one atmosphere), located outside of Earth's orbit, present in a car that is accelerating or decelerating, located in a flying airplane, or other locations that may have impacts on the accelerometer device. In such example embodiments, a user interface may be provided for the patient to provide input data (e.g., text, multiple choice, etc.) that delineates information that may cause the accelerometer device to provide different or anomalous data.

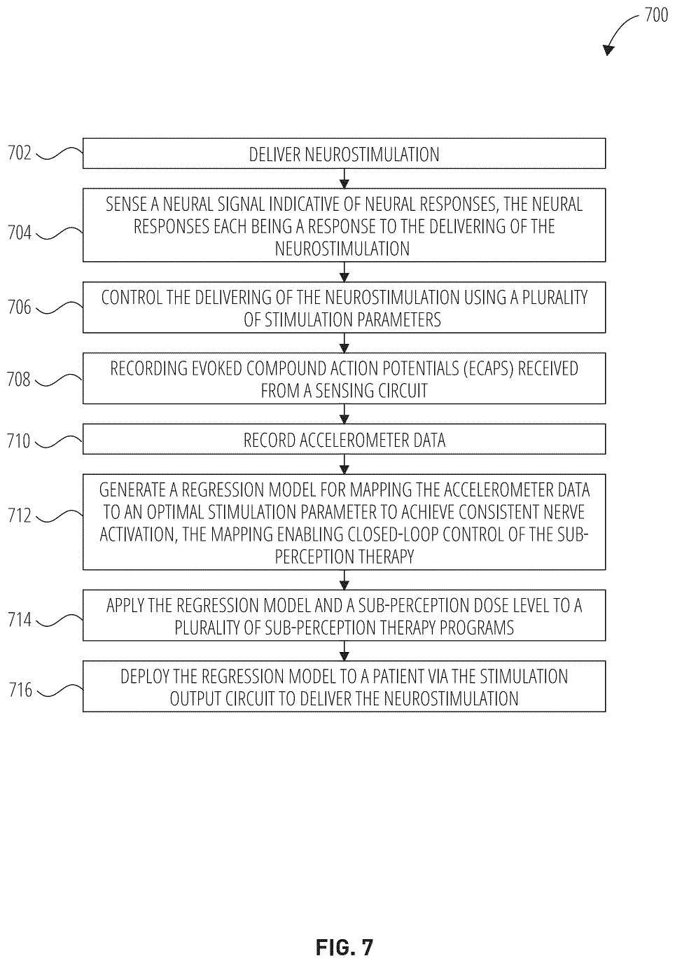

FIG. 7 illustrates a flowchart showing one example of a process flow 700 (e.g., processing method) for parameterizing closed-loop algorithms for sub-perception therapy, in accordance with example embodiments of the present disclosure. The embodiment of the process flow 700 can be implemented by a system or device for use to analyze, monitor, and correlate health-related data for neurostimulation programming (e.g., in connection with monitoring and recording evoked compound action potentials (ECAPs) and accelerometer data for use in closed-loop neurostimulation treatment and machine learning training and prediction models. For example, the process flow 700 can be embodied by electronic operations performed by one or more computing systems or devices (including those at a network-accessible remote service) that are specially programmed to implement data monitoring, data analysis, and/or neurostimulation data processing operations, and providing treatment and dose suggestions and changes (e.g., alterations) to patient systems. In specific examples, the operations of the process flow 700 can be implemented through the systems and data flows depicted throughout the disclosure, at a single entity or at multiple locations. For example, the process flow 700 can be performed by the monitoring and processing system 130.

In block 702, routine 700 delivers neurostimulation. In block 704, routine 700 senses a neural signal indicative of neural responses, the neural responses each being a response to the delivering of the neurostimulation. In block 706, routine 700 controls the delivering of the neurostimulation using a plurality of stimulation parameters. In block 708, routine 700 recording evoked compound action potentials (ECAPs) received from a sensing circuit. In block 710, routine 700 records accelerometer data. In block 712, routine 700 generates a regression model for mapping the accelerometer data to an optimal stimulation parameter to achieve consistent nerve activation, the mapping enabling closed-loop control of the sub-perception therapy. In block 714, routine 700 applies the regression model and a sub-perception dose level to a plurality of sub-perception therapy programs. In block 716, routine 700 deploys the regression model to a patient via the stimulation output circuit to deliver the neurostimulation.

Example embodiments of the present disclosure include the use of an accelerometer in conjunction with recorded neural signals to augment control algorithms for spinal cord stimulator (SCS) therapies or other stimulation therapies. Implantable devices, such as IPGs, sensor(s), or other devices, may be configured to sense nerve activity, such as evoked compound action potentials (ECAPs), and identify a neural activation index (NAI), which can include a score or quantity to describe how closely the system is to be achieving optimal or near-optimal neural activation consistency for a given patient. Example embodiments allow for closed loop algorithms based on ECAPs for all therapy modalities, independent of whether ECAPs are present at therapeutic doses.

In a first example embodiment, for a clinical workflow, a mapping from a sensor directly to one or more stimulation parameters in a paresthesia program includes a constant neural activation mapping that is used to fit an algorithm by applying a sub-perception program dose equal to, for example, 50% of a perception threshold in order to identify a stimulation amplitude. For example, a clinical workflow example can include collecting data (e.g., ECAP data and accelerometer data) from closed-loop paresthesia, confirming consistent perception. In such an example embodiment, the clinical workflow includes omitting actions that may cause changes in perception without significant changes in accelerometer (e.g., neck movement, coughs, sneezes, etc.) recordings. The example clinical workflow can include, if possible, a comprehensive set of movements so that accelerometer mapping is well-defined for all patient postures (e.g., standing, sitting, reclining, supine, lying left, lying right, leaning forward, etc.). The workflow continues to apply the mapping to all sub-perception programs desired for closed-loop (e.g., no additional closed-loop paresthesia required) by a clinician programmer and enabling the input for required doses for each program. The example clinical workflow further includes writing accelerometer-to-mapping and dose level suggestions (e.g., therapy changes) to IPG firmware or the like.

In example embodiments monitoring sub-paresthesia mode (e.g., fast mode) in patient therapies, there may be different sensors measuring what is felt and/or interpreted by a patient (e.g., how the patient interprets the sub-paresthesia mode). For example, when sub-paresthesia mode (e.g., below perception program) is applied to a patient, the patient is primarily intended not to interpret the applied stimulation (e.g., the user usually does not feel the applied therapy or program). However, in some circumstances, a patient can feel (or interpret the sub-paresthesia mode therapies) by, for example, raising the sub-paresthesia amplitude, rate, frequency, or the like. In such a circumstance, in a clinical workflow example, the system applies a mapping to one or more sub-perception programs, and a user (such as a clinician, or software model) can confirm that a patient is or is not feeling (e.g., interpreting) the sub-paresthesia mode, therapy, or program. Further example embodiments of the present disclosure can provide suggested adjustments, changes, variations, or the like to implement on the mapping according to sub-perception or perception-based programming.

In additional example embodiments, similar to the clinical workflow example embodiment, a system includes mapping from a sensor directly to a stimulation parameter that can be applied in an at-home scenario (e.g., without the need for clinician or doctor input or programming). For example, via a mobile application or remote control for receiving patient input, the application can collect data, such as recorded ECAP factors and recorded accelerometer data from closed-loop paresthesia monitoring. The at-home workflow can confirm consistent or approximately consistent perception from a patient and prompt the patient to move in a variety of positions or postures, such as sitting, leaning, bending, twisting sidewise, bathing movements, dressing movements, sleeping movements, or the like. The system is configured to monitor and flag or omit actions that may cause changes in perception without significant changes in accelerometer movement (e.g., neck movement, coughs, etc.). The example at-home workflow further includes, when possible, one or more comprehensive sets of movements that provide accelerometer mapping for all postures (e.g., standing, sitting, reclining, supine, lying left, lying right, leaning forward, etc.), some or all of which can be well-defined based on recorded patient data, exemplary data, fictionalized data, computer-rendered example data, or combinations thereof. The example workflow stores collected data in, for example, Ferroelectric RAM (FeRAM or FRAM), that is a random-access memory similar in construction to DRAM but using a ferroelectric layer instead of a dielectric layer to achieve non-volatility. The example at-home workflow can perform model training on IPG (if not computationally, resource, and/or power expensive), or, if more computing power is required, can stream data off an IPG to a mobile application and/or computing system for training and prediction modeling. The at-home workflow can apply mapping to all sub-perception programs desired for closed-loop by a programmer, and input required doses for each program to be applied at a per-patient level. The at-home workflow can further write accelerometer-mapping data and dose level data to IPG firmware, such as SRAM.

In an additional example embodiment, systems are configured for augmenting ECAP control algorithms with accelerometer input. For example, accelerometer mappings to stimulation control can be trained to have utility beyond sub-perception. For example, with paresthesia closed loop (CL) applications, the accelerometer mapping yields another possible control action to take, in addition to the ECAP feedback adjustment rule. A weighted combination of algorithm outputs may deliver an optimal (e.g., best, optimized, encouraged, desired, etc.) outcome:

CL-outputtotal=α*CL-outputECAP+β*CL-outputaccelerometer

Where α and β denote the relative weights given to an ECAP algorithm and accelerometer algorithm, respectively (sum to 1). For combination therapy, such as CL applications, the ECAP signal can be available less frequently than during paresthesia CL (due to other programs running, possible arbitration considerations, etc.). As such, the accelerometer mapping and/or output may be given greater weight in the above equation to compensate for less information from the ECAP signal. Additional utilities include anomaly detection: if CL-outputECAP and CL-outputaccelerometer are substantially different over an extended period of time (e.g., minutes or longer), then the anomaly detection likely determines either an IPG pocket or lead position/impedance has significantly changed. In such anomalous detection scenarios, a patient or user may be prompted to follow the example at-home (e.g., mobile) workflow to reformulate accelerometer mapping. An anomaly detector can be configured to identify divergent outputs and account for changes in values.

In another example embodiment, performance metrics for closed loop algorithms including accelerometer data based on ECAP control is provided. In such example embodiments, metrics can be used to quantify therapy delivery and algorithm performance in closed-loop applications according to some example embodiments. A neural activation index (NAI) can include a quantity to describe how closely the system is to be achieving optimal neural activation consistency. NAI=1, means completely consistent neural activation (optimal). NAI=0 means completely inconsistent neural activation (worst-case, least optimal).

For example, a demonstration of ECAP CL control can be derived from a variety of data, such as:
ParesthesiaNAI=variance around static posture, static stimulation amplitude/variance around CL setpoint (Paresthesia CL), or
Combo/Sub-P NAI=Paresthesia NAI*correlation (Combo/Sub-P mapping, Paresthesia mapping).

In additional example embodiments, migration of IPG pocket or migration of leads can include re-mapping and/or re-programming. Mobility-challenged patients may have difficulty helping to record all necessary postural data (e.g., various postures needed or preferred to program examples of the algorithms used by the system). User input can be used to identify varying postures a patient is unable to assume or have difficulty assuming (e.g., a 0 to 10 scale of difficulty in a given posture).

While the ECAP factors (e.g., features) and neural response parameters illustrated in FIG. 7 are discussed as examples, other ECAPs and neural response parameters can be suitable and used in the present subject matter as determined by those skilled in the art, for example, such as dynamic curves lengths, time-domain parameters, spectral power parameters, or the like.

FIG. 8 illustrates, by way of example, an embodiment of an implantable device 801-N and a lead system 805, such as may be implemented in a neuromodulation system according to some example embodiments of the present disclosure. The implantable device 802 may represent an example of the programming device 1202 and/or the stimulation device 1204 as described and depicted in connection with FIG. 12.

The implantable device 802 may include a controller 806 for use in controlling various functions of the implantable device. The implantable device 802 may be configured to sense ECAPs and to automatically select and maintain ECAP-sensing electrodes for use in sensing the ECAPs over an extended period of time.

The implantable device 801-N may include a stimulation output circuit 807 and the controller 806 may include a stimulation control circuit 808 configured for controlling the stimulation output circuit 807. The stimulation output circuit 807 may produce and deliver a neuromodulation waveform. Such waveforms may include different waveform shapes. The waveform shapes may include regular shapes (e.g., square, sinusoidal, triangular, saw tooth, and the like) or irregular shapes. The stimulation control circuit 808 may control which electrodes are used to deliver stimulation and may control the delivery of the neuromodulation waveform using the plurality of stimulation parameters, which specifies a pattern of the neuromodulation waveform. The lead system 805 may include one or more leads each configured to be electrically connected to stimulation device and a plurality of electrodes distributed in the one or more leads.

In an example, the number of leads and/or the number of electrodes on each lead depend on, for example, the distribution of target(s) of the neuromodulation and the need for controlling the distribution of electric field at each target. In an example, the lead system includes, for example, 19 leads each having 8 electrodes. The plurality of electrodes may include electrode 801-1, electrode 801-2, electrode 801-3 . . . and electrode 801-N. The implantable device 802, such as an IPG or ETS, may individually select electrodes to be active to provide an electrical interface between the stimulation output circuit 807 and the tissue of the patient. The neuromodulation waveform may be delivered from stimulation output circuit 807 through a set of active electrodes selected from electrodes 801-1 through 801-N.

The implantable device 801-N may include an ECAP sensing circuit 809 and the controller 806 may include an ECAP sensing control circuit 810 configured for controlling the ECAP sensing circuit 809. The implantable device 802 may be configured to individually select electrodes for use to sense electrical activity in neural tissue. The ECAP sensing circuit may include amplifiers and filters for use to detect the ECAPs in the neural tissue. The ECAP sensing control circuit 810 may control the electrodes that are used to sense the ECAPs and may also be configured to perform the evaluation of the sensing-capable electrodes in the lead system 805, which will be described in more detail below.

The controller 806 may further include an ECAP analyzer 811 configured to evaluate the detected ECAPs. By way of example and not limitation, various features in the detected ECAPs may be used to control a therapy and/or monitor an efficacy of the therapy. By way of example and not limitation, the present subject matter may use characteristics of the sensed ECAPs, such as low amplitude or a significant change in detected ECAP amplitude compared to a threshold or trend, to trigger the evaluation of sensing-capable electrodes. By way of example and not limitation, the implantable device 802 may include a scheduler 812, which either may or may not form part of the controller 806, to provide a programmed schedule for triggering the evaluation of sensing-capable electrodes. By way of example and not limitation, the implantable device 802 may include other sensing circuit(s) 813 to interface with other sensor(s) 814. By way of example, these sensor(s) may be used to control a therapy and/or monitor an efficacy of the therapy.

These sensor(s) may be used to trigger the evaluation of sensing-capable electrodes. For example, the sensor(s) may be used to detect significant patient activity or motion or posture changes, and the evaluation of sensing-capable electrodes may be triggered based at least in part on the detected activity, motion, or posture. For sensor(s) used to monitor the efficacy of the therapy, the system may be configured to trigger the evaluation of sensing-capable electrodes when the monitored efficacy of therapy is worse than expected or is trending lower. The implantable device may include a power source 815, such as a rechargeable battery or a passive energy source configured to receive power from an external device and may further include telemetry 816 for communicating with an external device, such as the stimulation device 1204 as described and depicted in connection with FIG. 12.

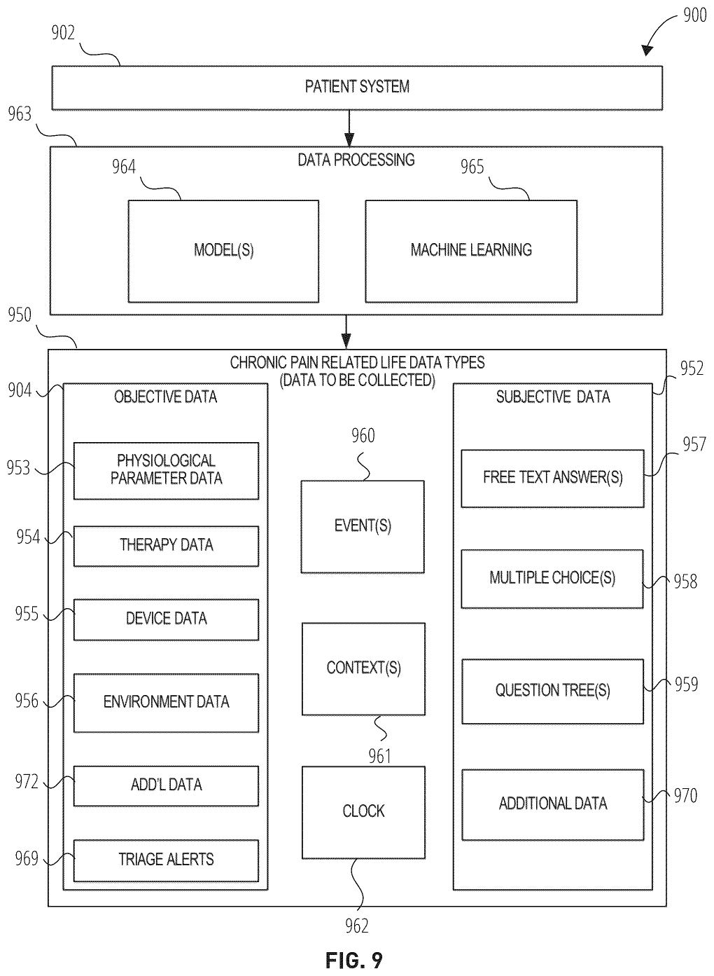

FIG. 9 illustrates a block diagram 900, by way of example and not limitation, including a patient system 902 for monitoring parameterizing techniques for sub-perception therapy and multi-sensor paresthesia therapy and configured to determine, from the health-related information, that patient issue(s) can be adversely affecting the patient treatment plan. The monitor can collect different chronic pain related life data types 950, which can include, for example, objective data 904, and/or subjective data 952.

Objective data is data that can be observed using human senses and can be obtained from a measurement or direct observation. Objective data can be measured by a sensor and can be provided via user input when the user has access to objectively determined information. Examples of objective data can include physiological parameter data 953, therapy data 954, device data 955, environment data 956, and/or additional objective data 972 (e.g., health related data). By way of example and not limitation, physiological parameter data 953 can include data such as: heart rate, blood pressure, respiration rate, activity, posture, electromyograms (EMGs), neural responses such as evoked compound action potentials (ECAPs), glucose measurements, oxygen levels body temperature, oxygen saturation, and gait. By way of example and not limitation, therapy data 954 can include: neuromodulation programs, therapy on/off schedule, dosing, neuromodulation parameters such as waveform, frequency, amplitude, pulse width, period, therapy usage and therapy type. By way of example and not limitation, device data 955 can include: battery information (voltage, charge state, charging history if rechargeable), impedance data, faults, device model, lead models, MRI status, Bluetooth connection logs, connection history with Clinician's Programmer (CP). By way of example and not limitation, environment data 956 can include: temperature, air quality, pressure, location, altitude, sunny, cloudy, precipitation, etc. By way of example, additional data 958 can include: healthcare-related data (e.g., menstrual cycles, surgeries, procedures, acute conditions, other non-pain chronic conditions, etc.), lifestyle-related data (e.g., relationship problems, financial problems, at-home stressors, at-work stressors, etc.), and the like.

Subjective data can include information received from the patient. For example, the patient's quantification of pain is a subjective data. Subjective data can generally involve user-inputted data. Examples of subjective data 952 include questions with free text answer(s) 957, multiple choice questions 958, question tree(s) 959, and/or additional subjective data 970. Other data can be stored and/or transferred, including detected event(s) 960, contextual data 963 (e.g., context(s) 961) for other collected data and/or event(s), and a clock 962 (e.g., time) such as can be used to provide a time stamp associated with the retrieved data. The event(s) 960, context(s) 961, and time can be detected by the system or can be provided via user input.

The collected data can be processed, as generally illustrated at data processing 963. The data processing 963 can occur in a medical device or a patient device such as a phone, tablet, or remote control, or can occur in a remote data receiving system. The data processing 963 can include one or more model(s) 964. The model(s) 964 can be used to determine how the patient data is used to determine issues (e.g., “common issues”) for which automatic triage alerts 969 can be beneficial to the user (e.g., the patient, the clinician, the device representative, the patient caregiver, etc.). The model(s) 964 can be used to determine how the patient data is used to classify the sub-perception therapy and multi-sensor paresthesia therapy (e.g., disease progression, updated patient goals, change of state, etc.) or the patient interaction, determine the type and contact of intervention, determine issues (e.g., “common issues”) for which automatic patient assistance can be beneficial, determine content for the patient assistance, determine if automatic triage alerts are beneficial, determine if treatment plan updates are beneficial, and the like. The model(s) 964 can use the acquired data to deliver therapy. Machine learning 965 (or other artificial intelligence) can be implemented on the collected data to develop or refine the model(s) 964. The data processing 963 can include data imputation such as can be used to prevent missing data from introducing bias into the model(s) or machine learning.

Broadly, machine learning may involve using computer algorithms to automatically learn patterns and relationships in data, potentially without the need for explicit programming. Machine learning algorithms can be divided into three main categories: supervised learning, unsupervised learning, and reinforcement learning.

For example, supervised learning involves training a model using labeled data to predict an output for new, unseen inputs. Examples of supervised learning algorithms include linear regression, decision trees, and neural networks. Unsupervised learning involves training a model on unlabeled data to find hidden patterns and relationships in the data. Examples of unsupervised learning algorithms include clustering, principal component analysis, and generative models like autoencoders. Reinforcement learning involves training a model to make decisions in a dynamic environment by receiving feedback in the form of rewards or penalties. Examples of reinforcement learning algorithms include Q-learning and policy gradient methods. Examples of specific machine learning algorithms that may be deployed, according to some examples, include logistic regression, which is a type of supervised learning algorithm used for binary classification tasks. Logistic regression models the probability of a binary response variable based on one or more predictor variables. Another example type of machine learning algorithm is Naïve Bayes, which is another supervised learning algorithm used for classification tasks. Naïve Bayes is based on Bayes' theorem and assumes that the predictor variables are independent of each other. Random Forest is another type of supervised learning algorithm used for classification, regression, and other tasks. Random Forest builds a collection of decision trees and combines their outputs to make predictions.

Further examples include neural networks, which consist of interconnected layers of nodes (or neurons) that process information and make predictions based on the input data. Matrix factorization is another type of machine learning algorithm used for recommender systems and other tasks. Matrix factorization decomposes a matrix into two or more matrices to uncover hidden patterns or relationships in the data. Support Vector Machines (SVM) are a type of supervised learning algorithm used for classification, regression, and other tasks. SVM finds a hyperplane that separates the different classes in the data. Other types of machine learning algorithms include decision trees, k-nearest neighbors, clustering algorithms, and deep learning algorithms such as convolutional neural networks (CNN), recurrent neural networks (RNN), and transformer models. The choice of algorithm depends on the nature of the data, the complexity of the problem, and the performance requirements of the application. The performance of machine learning models is typically evaluated on a separate test set of data that was not used during training to ensure that the model can generalize to new, unseen data.

Although several specific examples of machine learning algorithms are discussed herein, the principles discussed herein can be applied to other machine learning algorithms as well. Deep learning algorithms such as convolutional neural networks, recurrent neural networks, and transformers, as well as more traditional machine learning algorithms like decision trees, random forests, and gradient boosting may be used in various machine learning applications.

Two example types of problems in machine learning are classification problems and regression problems. Classification problems, also referred to as categorization problems, aim at classifying items into one of several category values (e.g., is this object an apple or an orange?). Regression algorithms aim at quantifying some items (for example, by providing a value that is a real number).

Turning to the training phases in machine learning algorithms and models, generating a trained machine-learning program may include multiple phases that form part of a machine-learning pipeline, including for example the following phases: data collection and preprocessing, feature engineering, model selection and training, model evaluation, prediction, validation, refinement, retraining, deployment, or a combination thereof. For example, data collection and preprocessing can include a phase for acquiring and cleaning data to ensure that it is suitable for use in the machine learning model. This phase may also include removing duplicates, handling missing values, and converting data into a suitable format. Feature engineering can include a phase for selecting and transforming the training data to create features that are useful for predicting the target variable. Feature engineering may include (1) receiving features (e.g., as structured or labeled data in supervised learning) and/or (2) identifying features (e.g., unstructured, or unlabeled data for unsupervised learning) in training data. Model selection and training can include a phase for selecting an appropriate machine learning algorithm and training it on the preprocessed data. This phase may further involve splitting the data into training and testing sets, using cross-validation to evaluate the model, and tuning hyperparameters to improve performance.

In additional examples, model evaluation can include a phase for evaluating the performance of a trained model (e.g., the trained machine-learning program) on a separate testing dataset. This phase can help determine if the model is overfitting or underfitting and determine whether the model is suitable for deployment. Prediction can include a phase for using a trained model (e.g., trained machine-learning program) to generate predictions on new, unseen data. Validation, refinement, or retraining can include a phase for updating a model based on feedback generated from the prediction phase, such as new data or user feedback. Deployment can include a phase for integrating the trained model (e.g., the trained machine-learning program) into a more extensive system or application, such as a web service, mobile app, or IoT device. This phase can involve setting up APIs, building a user interface, and ensuring that the model is scalable and can handle large volumes of data.

Example phases of machine learning can further include a training phase (e.g., part of the model selection and trainings) and a prediction phase (part of prediction). Prior to the training phase, feature engineering is used to identify features. This may include identifying informative, discriminating, and independent features for effectively operating the trained machine-learning program in pattern recognition, classification, and regression. In some examples, the training data includes labeled data, known for pre-identified features and one or more outcomes. Each of the features may be a variable or attribute, such as an individual measurable property of a process, article, system, or phenomenon represented by a data set (e.g., the training data). Features may also be of different types, such as numeric features, strings, and graphs, and may include one or more of content, concepts, attributes, historical data, and/or user data, merely for example.

In training phase, the machine-learning pipeline uses the training data to find correlations among the features that affect a predicted outcome or prediction/inference data. With the training data and the identified features, the trained machine-learning program is trained during the training phase during machine-learning program training. The machine-learning program training appraises values of the features as they correlate to the training data. The result of the training is the trained machine-learning program (e.g., a trained or learned model). Further, the training phase may involve machine learning, in which the training data is structured (e.g., labeled during preprocessing operations). The trained machine-learning program implements a neural network capable of performing, for example, classification and clustering operations. In other examples, the training phase may involve deep learning, in which the training data is unstructured, and the trained machine-learning program implements a deep neural network that can perform both feature extraction and classification/clustering operations.

In some examples, a neural network may be generated during the training phase and implemented within the trained machine-learning program. The neural network includes a hierarchical (e.g., layered) organization of neurons, with each layer consisting of multiple neurons or nodes. Neurons in the input layer receive the input data, while neurons in the output layer produce the final output of the network. Between the input and output layers, there may be one or more hidden layers, each consisting of multiple neurons. Each neuron in the neural network operationally computes a function, such as an activation function, which takes as input the weighted sum of the outputs of the neurons in the previous layer, as well as a bias term. The output of this function is then passed as input to the neurons in the next layer. If the output of the activation function exceeds a certain threshold, an output is communicated from that neuron (e.g., transmitting neuron) to a connected neuron (e.g., receiving neuron) in successive layers. The connections between neurons have associated weights, which define the influence of the input from a transmitting neuron to a receiving neuron. During the training phase, these weights are adjusted by the learning algorithm to optimize the performance of the network. Different types of neural networks may use different activation functions and learning algorithms, affecting their performance on different tasks. The layered organization of neurons and the use of activation functions and weights enable neural networks to model complex relationships between inputs and outputs, and to generalize to new inputs that were not seen during training.

In some examples, the neural network may also be one of several different types of neural networks, such as a single-layer feed-forward network, a Multilayer Perceptron (MLP), an Artificial Neural Network (ANN), a Recurrent Neural Network (RNN), a Long Short-Term Memory Network (LSTM), a Bidirectional Neural Network, a symmetrically connected neural network, a Deep Belief Network (DBN), a Convolutional Neural Network (CNN), a Generative Adversarial Network (GAN), an Autoencoder Neural Network (AE), a Restricted Boltzmann Machine (RBM), a Hopfield Network, a Self-Organizing Map (SOM), a Radial Basis Function Network (RBFN), a Spiking Neural Network (SNN), a Liquid State Machine (LSM), an Echo State Network (ESN), a Neural Turing Machine (NTM), or a Transformer Network, merely for example.

In addition to the training phase, a validation phase may be performed on a separate dataset known as the validation dataset. The validation dataset is used to tune the hyperparameters of a model, such as the learning rate and the regularization parameter. The hyperparameters are adjusted to improve the model's performance on the validation dataset. Once a model is fully trained and validated, in a testing phase, the model may be tested on a new dataset. The testing dataset is used to evaluate the model's performance and ensure that the model has not overfitted the training data.

In a prediction phase, the trained machine-learning program uses the features for analyzing query data to generate inferences, outcomes, or predictions, as examples of a prediction/inference data. For example, during prediction phase, the trained machine-learning program generates an output. Query data is provided as an input to the trained machine-learning program, and the trained machine-learning program generates the prediction/inference data as output, responsive to receipt of the query data.

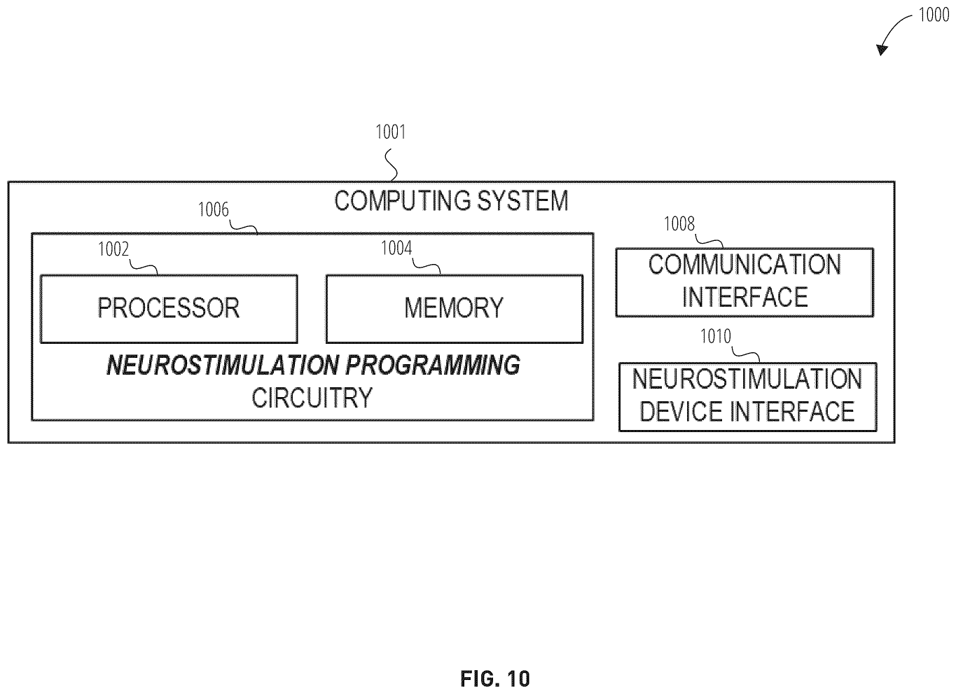

FIG. 10 illustrates, by way of example, a block diagram 1000 of an embodiment of a system 1001 (e.g., a computing system) implementing neurostimulation programming circuitry 1206 to cause programming of an implantable electrical neurostimulation device, for accomplishing the therapy objectives in a human subject based on a trained closed-loop programming model as discussed herein.

The system 1001 may be operated by a clinician, a patient, a caregiver, a medical facility, a research institution, a medical device manufacturer or distributor, and embodied in a number of different computing platforms. The system 1001 may be a remote control device, patient programmer device, program modeling system, or other external device, including a regulated device used to directly implement programming commands and modification with a neurostimulation device. In some examples, the system 1001 may be a networked device connected via a network (or combination of networks) to a computing system operating a user interface computing system using a communication interface 1008. The network may include local, short-range, or long-range networks, such as Bluetooth, cellular, IEEE 802.11 (Wi-Fi), or other wired or wireless networks.

The system 1001 includes a processor 1002 and a memory 1004, which can be optionally included as part of neurostimulation stimulation programming circuitry 1006. The processor 1002 may be any single processor or group of processors that act cooperatively. The memory 1004 may be any type of memory, including volatile or non-volatile memory. The memory 1004 may include instructions, which when executed by the processor 1202, cause the processor 1002 to implement the features of the stimulation programming circuitry 1006 (e.g., neurostimulation circuitry). Thus, the electronic operations in the system 1200 may be performed by the processor 1002 or the stimulation programming circuitry 1006.

The processor 1002 or circuitry 1006 may directly or indirectly implement neurostimulation operations including the use of neurostimulation device programming based on a trained programming model. The processor 1002 or circuitry 1006 may further provide data and commands to assist the processing and implementation of the programming using communication interface 1008 or a stimulation device interface 1010 (e.g., neurostimulation interface). It will be understood that the processor 1002 or circuitry 1006 may also implement other aspects of the device data processing or device programming functionality described above.

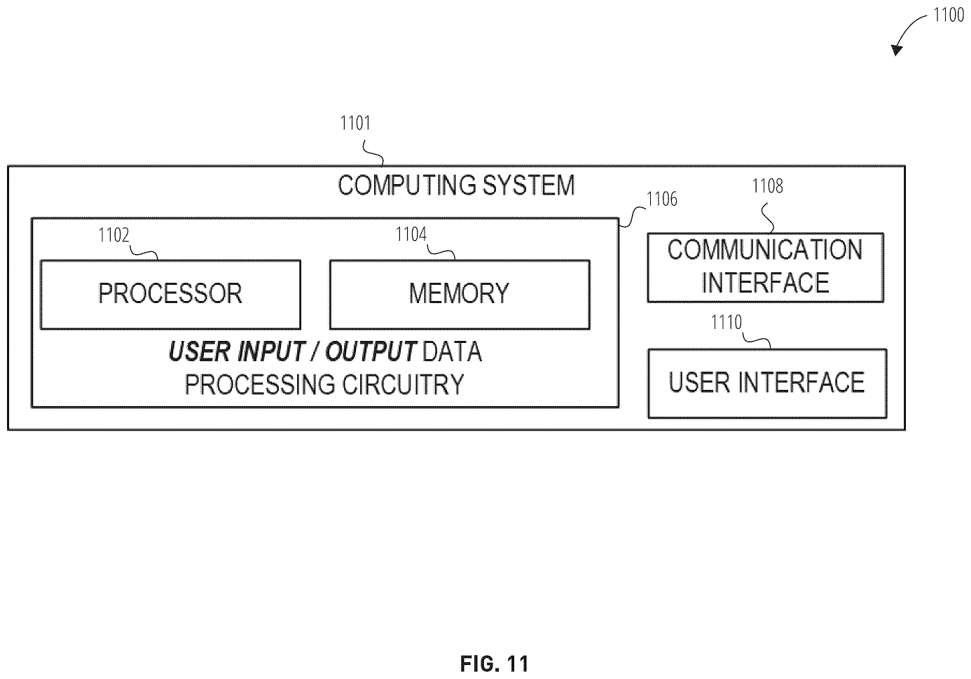

FIG. 11 illustrates, by way of example, a block diagram 1100 of an embodiment of a system 1101 (e.g., a computing system) for performing analysis of patient feedback data (e.g., patient input data collected as training data in a user stimulation device interface 1110) in connection with the data processing operations discussed above.

The system 1101 may be integrated with or coupled to a computing device, a remote-control device, patient programmer device, clinician programmer device, program modeling system, or other external device, deployed with neurostimulation treatment. In some examples, the system 1101 may be a networked device (server) connected via a network (or combination of networks) which communicates to one or more devices (clients) using a communication interface 1108 (e.g., communication hardware which implements software network interfaces and services). The network may include local, short-range, or long-range networks, such as Bluetooth, cellular, IEEE 802.11 (Wi-Fi), or other wired or wireless networks.

The system 1101 includes a processor 1102 and a memory 1104, which can be optionally included as part of a stimulation programming circuitry 1106 (e.g., user input/output data processing circuitry). The processor 1102 may be any single processor or group of processors that act cooperatively. The memory 1104 may be any type of memory, including volatile or non-volatile memory. The memory 1104 may include instructions, which when executed by the processor 1102, cause the processor 1102 to implement data processing, or to enable other features of the user input/output data processing stimulation programming circuitry 1106. Thus, electronic operations in the system 1101 may be performed by the processor 1102 or the circuitry 1106.

For example, the processor 1102 or the circuitry 1106 may implement any of the features of the method 800 to obtain and process patient feedback data, to generate user interface displays, and to provide training data for training of a programming selection model. It will be understood that the processor 1102 or the circuitry 1106 may also implement aspects of the logic and processing described above, for use in various forms of open-loop, closed-loop, partially-closed-loop device programming or related device actions.

FIG. 12 illustrates a block diagram 1200 of an embodiment of a neurostimulation system. The block diagram 1200 includes electrodes 1206, a stimulation device 1204, and a programming device 1202. Electrodes 1206 are configured to be placed on or near one or more neural targets in a patient. Stimulation device 1204 is configured to be electrically connected to electrodes 1206 and deliver neurostimulation energy, such as in the form of electrical pulses, to the one or more neural targets though electrodes 1206. The delivery of the neurostimulation is controlled by using a plurality of stimulation parameters, such as stimulation parameters specifying a pattern of the electrical pulses and a selection of electrodes through which each of the electrical pulses is delivered.

In various embodiments, at least some parameters of the plurality of stimulation parameters are selected or programmable by a clinical user, such as a physician or other caregiver who treats the patient using system 1300; however, some of the parameters may also be provided in connection with closed-loop programming logic and adjustment. Programming device 1202 provides the user with accessibility to implement, change, or modify the programmable parameters. In various embodiments, programming device 1220 is configured to be communicatively coupled to stimulation device 1204 via a wired or wireless link.

In various embodiments, programming device 1202 includes a user interface 1210 (e.g., a user interface embodied by a graphical, text, voice, or hardware-based user interface) that allows the user to set and/or adjust values of the user-programmable parameters by creating, editing, loading, and removing programs that include parameter combinations such as patterns and waveforms. These adjustments may also include changing and editing values for the user-programmable parameters or sets of the user-programmable parameters individually (including values set in response to a therapy efficacy indication). Such waveforms may include, for example, the waveform of a pattern of neurostimulation pulses to be delivered to the patient as well as individual waveforms that are used as building blocks of the pattern of neurostimulation pulses. Examples of such individual waveforms include pulses, pulse groups, and groups of pulse groups. The program and respective sets of parameters may also define an electrode selection specific to each individually defined waveform.

The present approaches further provide examples of an evaluation system 1212, such as a data analysis system, which is used to adapt, modify, start, stop, monitor, or identify a neurostimulation treatment with stimulation device 1204. This evaluation system 1212 initiates an action related to the neurostimulation treatment based on text analysis performed on input 1220 (e.g., text). This input text 1220 can be directly collected from the patient and analyzed by the evaluation system 1212, to then cause a programming effect in the programming device 1202, and the stimulation device 1204, and the neurostimulation treatment provided by the electrodes 1206.

A user (e.g., the patient, clinician, device representative, software, etc.) can provide parameter inputs to the evaluation system 1212, which are used to select, load, modify, implement, measure, analyze, monitor, and/or evaluate one or more parameters of a defined program for neurostimulation treatment that is implemented by the stimulation device 1204, or the operation of the stimulation device 1204. This evaluation can be based on a combination of natural language processing, sentiment analysis, rules, and other operational or treatment objectives that are identified. Various logic or algorithms can then determine an appropriate action to take based on the state of the patient, including but not limited to: a program or parameter change or recommendation to produce an improvement for a treatment objective (such as to address pain, increase mobility, reduce sleep disruption, and the like); diagnostic or remedial actions on the stimulation device 1304; data logging or alerts to the patient or a clinician associated with the patient; and the like.

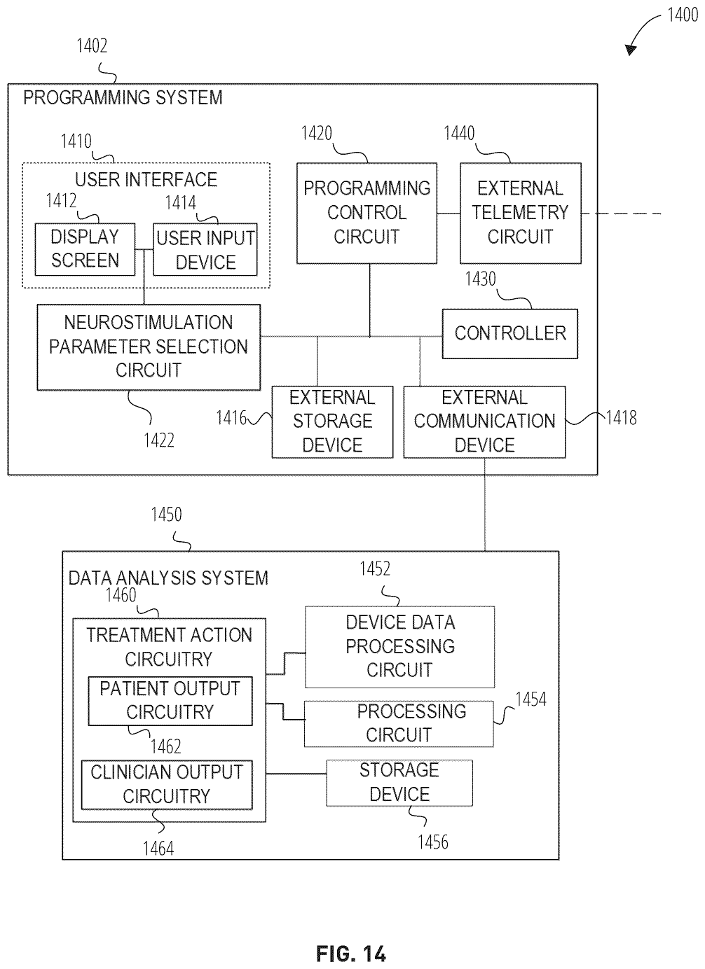

Example parameters that can be implemented by a selected neurostimulation program include, but are not limited to the following: amplitude, pulse width, frequency, duration, total charge injected per unit time, cycling (e.g., on/off time), pulse shape, number of phases, phase order, interphase time, charge balance, ramping, as well as spatial variance (e.g., electrode configuration changes over time). As detailed in FIG. 14, a controller, e.g., controller 1430 of FIG. 14, can implement program(s) and parameter setting(s) to affect a specific neurostimulation waveform, pattern, or energy output, using a program, or setting in storage (e.g., external storage device 1416 of FIG. 14), or using settings communicated via an external communication device 1418 of FIG. 14 corresponding to the selected program. The implementation of such program(s) or setting(s) may further define a therapy strength and treatment type corresponding to a specific pulse group, or a specific group of pulse groups, based on the specific programs or settings. The evaluation system 1212 and the evaluation of the input 1220 (e.g., text, signal, visual, etc.) provides a mechanism to determine the effectiveness of such programs or settings, and to identify issues and provide remediation for ineffective programs or settings, offer suggestions or recommendations for new or updated programs and/or settings, or even to automatically change programs or settings.

Portions of the evaluation system 1212, the stimulation device 1204 (e.g., implantable medical device, attachable device, etc.), or the programming device 1202 can be implemented using hardware, software, or any combination of hardware and software. Portions of the stimulation device 1204 or the programming device 1202 can be implemented using an application-specific circuit that can be constructed or configured to perform one or more particular functions or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. The neuromodulation system as shown in block diagram 1200 could also include a subcutaneous medical device (e.g., subcutaneous implantable cardioverter-defibrillator (S-ICD), subcutaneous diagnostic device, wearable medical devices (e.g., patch-based sensing device), or other external medical devices.

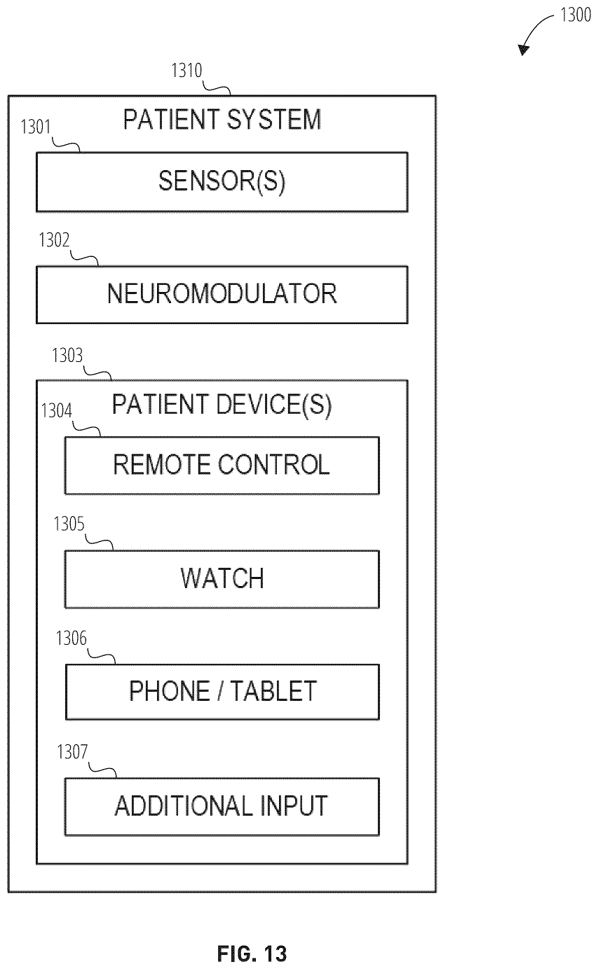

FIG. 13 illustrates a block diagram 1300 of a patient system 1310, and examples of devices that may make up components of the patient system, in accordance with some example embodiments. The patient system 1310 may include component(s) that act on the patient such as deliver a therapy to the patient, component(s) used to sense a condition, status or environment of the patient, and component(s) enabling the patient to interface with the patient system.

For example, the illustrated patient system patient system 1310 may include sensor(s) 1301 to sense patient parameter(s). Examples of sensors may include, but not limited to, neural activity, muscle movement, patient activity, patient posture, patient location, breathing, heart rate, blood pressure, temperature, analyte sensors and the like. The illustrated patient system patient system 1310 may include a therapy-delivering device such a neuromodulator 1302 configured to deliver a neuromodulation therapy such as a DBS, SCS, PNS, FES, transcutaneous electrical nerve stimulation (TENS), or other therapy. Those of ordinary skill in the art would understand, upon reading and comprehending the present disclosure, how to apply the present subject matter to other therapies, such as but not limited to cardiac rhythm therapies or drug pump therapies.

The illustrated patient system 1310 patient system 1310 may also include patient device(s) 1303 used by the patient to interface with the patient system. These device(s) may include sensor(s) (e.g., accelerometer, temperature sensor, heart rate sensor, etc.) and/or can be used to receive patient feedback such as responses to questions or free text responses. These device(s) may include interfaces used to interact with the therapy delivery. Examples of patient device(s) 1303 include, but are not limited to, a patient remote control 1304, a wearable device(s) such as a watch 1305, or a phone or tablet 1306, or another personal device or additional input 1307.

FIG. 14 illustrates a block diagram 1400 of a programming system 1402 used as part of an implantable neurostimulation system, such as the external system 1714, with the programming system 1402 configured to send and receive device data (e.g., commands, parameters, program selections, information), in accordance with example embodiments. FIG. 14 also illustrates an embodiment of a data analysis computing system 1450, communicatively coupled to the programming system 1402, with the data analysis computing system 1450 used to perform data analysis on freeform text and device data (or other types of data) in connection with neurostimulation treatment by the implantable neurostimulation system.

The programming system 1402 represents an embodiment of the programming device 1202 of FIG. 12, and includes an external telemetry circuit 1440, an external storage device 1416, a programming control circuit 1420, a user interface (UI) device 1410, a controller 1430, and an external communication device 1418, to effect programming of a connected neurostimulation device. The operation of the neurostimulation parameter selection circuit 1422 enables selection, modification, and implementation of a particular set of parameters or settings for neurostimulation programming. The particular set of parameters or settings that are selected, modified, or implemented can be based on the parameters, such as described and depicted with reference to the in FIGS. 1-7.

The external telemetry circuit 1440 provides the closed loop programming system 1402 with wireless communication to and from another controllable device such as the implantable stimulator including transmitting one or a plurality of stimulation parameters (e.g., selected, identified, or modified stimulation parameters of a selected program) to the implantable stimulator, via programming data 1560. In one embodiment, the external telemetry circuit 1440 also transmits power to the stimulator, such as implantable stimulator 1521 as described and depicted in connection with FIG. 15, through inductive coupling or the like.

The external communication device 1518 can provide a mechanism to conduct communications with a programming information source, such as a data service, program modeling system, to receive program information, settings and values, models, functionality controls, or the like, via an external communication link (not shown). In a specific example, the external communication device 1518 communicates with the data analysis system 1550 (e.g., such as a computing system) to obtain commands or instructions in connection with parameters or settings that are selected, modified, or implemented based on freeform text analysis from the data analysis system 1450. The external communication device 1418 may communicate using any number of wired or wireless communication mechanisms described in this document, including but not limited to IEEE 802.11 (Wi-Fi), Bluetooth, Infrared, and like standardized and proprietary wireless communications implementations. Although the external telemetry circuit 1440 and the external communication device 1418 are depicted as separate components within the programming system 1402, the functionality of both of these components can be integrated into a single communication chipset, circuitry, or device.

The external storage device 1416 stores a plurality of existing neurostimulation waveforms, including definable waveforms for use as a portion of the pattern of the neurostimulation pulses, settings and setting values, other portions of a program, and related treatment efficacy indication values. In various embodiments, each waveform of the plurality of individually definable waveforms includes one or more pulses of the neurostimulation pulses and may include one or more other waveforms of the plurality of individually definable waveforms. Examples of such waveforms include pulses, pulse blocks, pulse trains, and train groupings, and programs. The existing waveforms stored in the external storage device 1416 can be definable at least in part by one or more parameters including, but not limited to the following: amplitude, pulse width, frequency, duration(s), electrode configurations, total charge injected per unit time, cycling (e.g., on/off time), waveform shapes, spatial locations of waveform shapes, pulse shapes, number of phases, phase order, interphase time, charge balance, and ramping.

The external storage device 1416 may also store a plurality of individually definable fields that can be implemented as part of a program. Each waveform of the plurality of individually definable waveforms is associated with one or more fields of the plurality of individually definable fields. Each field of the plurality of individually definable fields is defined by one or more electrodes of the plurality of electrodes through which a pulse of the neurostimulation pulses is delivered and a current distribution of the pulse over the one or more electrodes. A variety of settings in a program can be correlated to the control of these waveforms and definable fields.

The programming control circuit 1420 represents an embodiment of a control circuit and can translate or generate the specific stimulation parameters or changes which are to be transmitted to the implantable stimulator, based on the results of the neurostimulation parameter selection circuit 1422. The pattern can be defined using one or more waveforms selected from the plurality of individually definable waveforms (e.g., defined by a program) stored in an external storage device 1416. In various embodiments, the programming control circuit 1420 checks values of the plurality of stimulation parameters against safety rules to limit these values within constraints of the safety rules. In one embodiment, the safety rules are heuristic rules.

The user interface device 1410 represents an embodiment of user interface devices and allows the user (e.g., a patient, representative, clinician, etc.) to provide input relevant to therapy objectives, such as to switch programs or change operational use of the programs. The user interface device 1410 includes a display screen 1412, a user input device 1414, and can implement or couple to the parameter selection circuit 1422, or data provided from the data analysis computing system 1450. The display screen 1412 can include any type of interactive or non-interactive screens, and the user input device 1414 can include any type of user input devices that supports the various functions discussed in this document, such as a touchscreen, keyboard, keypad, touchpad, trackball, joystick, mouse, physical human interaction (e.g., fingers, voice, sound, etc.), or the like. The user interface device 1410 may also allow the user to perform other functions where user interface input is suitable (e.g., to select, modify, enable, disable, activate, schedule, or otherwise define a program, sets of programs, provide feedback, or input values, or perform other monitoring and programming tasks). Although not shown, the user interface device 1410 may also generate a visualization of such characteristics of device implementation or programming and receive and implement commands to implement or revert the program and the neurostimulator operational values (including a status of implementation for such operational values). These commands and visualization can be performed in a review and guidance mode, status mode, or in a real-time programming mode.

The controller 1430 can be a microprocessor that communicates with the external telemetry circuit 1440, the external communication device 1418, the external storage device 1416, the programming control circuit 1420, the parameter selection circuit, and the user interface device 1410, via a bidirectional data bus or the like. The controller 1430 can be implemented by other types of logic circuitry (e.g., discrete components or programmable logic arrays) using a state machine type of design. As used in this disclosure, the term “circuitry” should be taken to refer to discrete logic circuitry, firmware, to the programming of a microprocessor, or a combination thereof.

The data analysis system 1450 is configured to operate treatment action circuitry 1460, which can produce or initiate certain actions on the basis of device data (received and processed by device data processing circuit 1452) and input text (received and processed by processing circuit 1454). The treatment action circuitry 1460 can identify one or more actions related to the neurostimulation treatment and provide outputs to a patient or a clinician using patient output circuitry 1462 or clinician (or other non-patient person, entity, program, or combination thereof) output circuitry 1464, respectively. Such outputs and actions provided by the outputs are based on the evaluation and detection of particular patient data, triage data, parameter data, device states from text and associated device data, or a combination thereof.

The data analysis system 1450 also is depicted as including a storage device 1456 to store or persist data related to the device data, text input, patient, clinician output, and related settings, logic, or algorithms. Other hardware features of the data analysis computing system 1450 are not depicted for simplicity but are suggested from functional capabilities and operations in the following figures.

As will be understood, patients who are experiencing chronic pain are often willing to provide detailed information regarding their current medical state within varying input options (e.g., freeform text answers to questions, questionnaires, question trees, associated applications, adaptation device data, etc.). For example, freeform text in the form of a narrative, explanatory statement, or interjection is easy for patients to produce, and can provide many details regarding a patient's actions, physiological, physical, and psychological state, prior historical events, and can reflect both objective and subjective results of neurostimulation treatment. Freeform text, however, can be time-consuming or difficult for physicians and clinicians to interpret, especially when patient feedback can be contradictory (e.g., “I felt good in the morning but was unable to do any activity”) or is incomplete without additional context (e.g., “I was unable to get out of bed”). Capturing patient feedback with the present systems can provide many new data points for treatment outcomes, can triage patients using alert notifications, and provide a basis for determining whether or why a particular neurostimulation treatment (and treatment program, programming value, programming effect) is or is not effective.

FIG. 15 illustrates a block diagram 1500, by way of example, an embodiment of data interactions among a monitoring and processing system implemented as a programming data service 1570, for operation of a stimulation device 1521 with use of closed loop programming, open loop programming, partially closed loop programming, or a combination thereof. At a high level, a programming data service 1570 uses a trained model (one of programming models 1502) to generate programming settings and parameters (e.g., in one or more programs 1505) that are customized to the patient. The programming data service 1570 includes compute hardware 1503 to control the model training 1501 and other data processing operations, such as to generate or control diagnostic actions, alerts, programming recommendations, or programming actions. The programming settings and parameters may be implemented automatically or manually on the stimulation device 1521 (e.g., using the programming techniques referenced above) or via a patient computing device 1520 or patient programming device 1530.

The programming data service 1570 communicates with one or both of the patient computing and programming devices 1520, 1530, via a network 1510 to obtain training data for use by model training logic 1501. The training data may be stored in a database 1504 or another large-scale data store (e.g., data lake) for the patient or a population of patients. The programming data service 1570 may also include data analysis or processing engines (not shown) that parse and determine a state of a particular patient from various inputs and correlated program usage (e.g., to determine what programs and programming settings are beneficial or not beneficial to the patient). In some examples, the state of treatment may be based on correlating the historical use of a neurostimulation program or set of parameters with the current state of a patient (e.g., identifying that a pain condition became worse or better after beginning use of a particular program).

The programming data service 1570 can also analyze a variety of forms of patient input and patient data related to usage of a neurostimulation program or neurostimulation programming parameters. For instance, the programming data service 1570 can receive information from program usage, questionnaire selections, text input originating from a human patient, and the like via the patient computing and programming devices 1520, 1530. In addition to providing recommended programs, the programming data service can also provide therapy content, triage alerts, and usage recommendations to the patient computing and programming devices 1520, 1530.

A patient can provide training data (e.g., input data) via the patient computing device 1520 or the patient programming device 1530. Additional detail of how input data is collected is discussed with reference to the data processing logic and user interfaces discussed in related patent application. In an example, the patient computing device 1520 is a computing device (e.g., personal computer, tablet, smartphone) or other form of user-interactive device which receives and provides interaction with a patient using a graphical user interface 1523, with use of programming input logic 1524 and programming output logic 1522. For instance, the programming input logic 1524 can receive input from a patient via questionnaires, surveys, messages, or other inputs. The inputs may provide text related to pain or overall health, which can be used to identify a psychological state(s), physical state(s), physiological state(s), somatic state(s), or a combination of states of the patient, neurostimulation treatment results, or related conditions. As used herein, the terms “neurostimulator,” “stimulator,” “neurostimulation,” and “stimulation” generally refer to the delivery of electrical energy that affects the neuronal activity of neural tissue, which may be excitatory or inhibitory; for example by initiating an action potential, inhibiting or blocking the propagation of action potentials, affecting changes in neurotransmitter/neuromodulator release or uptake, and inducing changes in neuro-plasticity or neurogenesis of tissue. It will be understood that other clinical effects and physiological mechanisms may also be provided through use of such stimulation techniques.

A patient programming device 1530 is depicted as including a user interface 1531 and program implementation logic 1532. The program implementation logic 1532 specifically can provide the patient with the ability to implement or switch to particular programs generated by programming data service 1570. Other forms of programming can also include the receipt of instructions, recommendations, or feedback (including clinician recommendations, behavioral modifications, etc., selected for the patient) that are automatically selected based on detected conditions.

The programming data service 1570 can also utilize sensor data 1540 from one or more patient sensors 1550 (e.g., wearables, sleep trackers, motion tracker, implantable devices, etc.) among one or more internal or external devices. The sensor data 1540 can be used to determine a customized and current parameterizing technique for sub-perception therapy and multi-sensor paresthesia therapy or neurostimulation treatment results. In various examples, the stimulation device 1521 also includes sensors that contribute to the sensor data 1540 to be evaluated by the programming data service 1570.

In an example, the patient sensors 1550 are physical, physiological, biopsychosocial, or similar sensors that collect data relevant to physical, biopsychosocial (e.g., stress and/or mood biomarkers), or physiological factors relevant to a state of the patient. Examples of such sensors might include a sleep sensor to sense the patient's sleep state (e.g., for detecting lack of sleep), a respiration sensor to measure patient breathing rate or capacity, a movement sensor to identify an amount or type of movement, a heart rate sensor to sense the patient's heart rate, a blood pressure sensor to sense the patient's blood pressure, an electrodermal activity (EDA) sensor to sense the patient's EDA (e.g., galvanic skin response), a facial recognition sensor to sense the patient's facial expression, a voice sensor (e.g., microphone) to sense the patient's voice, and/or an electrochemical sensor to sense stress biomarkers from the patient's body fluids (e.g., enzymes and/or ions, such as lactate or cortisol from saliva or sweat). Other types or form factors of sensor devices may also be utilized.

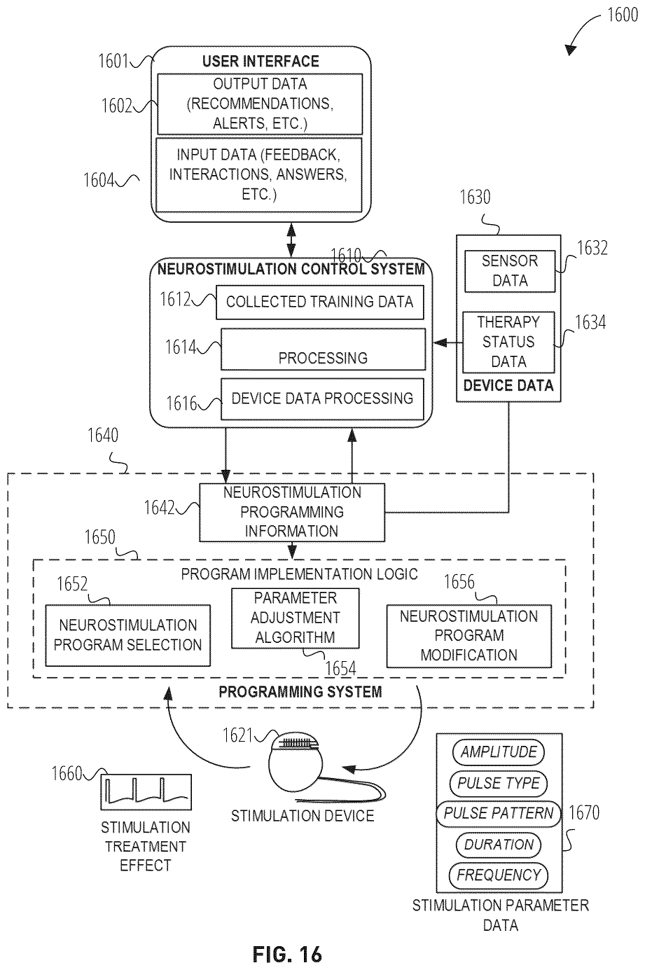

FIG. 16 illustrates, by way of example, an embodiment of a data processing flow 1600 affecting the neurostimulation treatment of a patient, including a neurostimulation control system 1610 based on collected training data 1612, data processing 1614, and device data processing 1616 functions. Here, additional details are provided on the data flow between the monitoring and processing system 130 and an example user interface 1601. Other user interfaces and actions are not depicted for simplicity.

In this example, input data 1604 (e.g., parameter input, questionnaire answers, question trees, freeform text, patient feedback, interaction results, etc.) is obtained by the monitoring and processing system 130. FIG. 16 also depicts the evaluation of device data 1630, such as sensor data 1632, therapy status data 1634, and other treatment aspects which can be obtained or derived from the neurostimulation device or related neurostimulation programming. Also in this example, output data 1602 (e.g., content) is obtained from the monitoring and processing system 130 at a user interface, such as in the form of recommendations, alerts, triage messages, imputation suggestions, treatment updates, patient information, and the like. The monitoring and processing system 130 can separately provide clinician recommendations, clinician alerts, or other related actions that are provided to the clinician, doctor, or device representative separately from the patient.

The remainder of the data processing flow 1600 illustrates how data processing results from the monitoring and processing system 130 can be used to effect programming, such as in a closed loop (or partially-closed-loop, open-loop, or other) system. A programming system 1640 can use parameters or programming information 1642 (e.g., program neurostimulation programming information) provided from the monitoring and processing system 130 as an input to program implementation logic 1650. The program implementation logic 1650 can be implemented by a parameter adjustment algorithm 1654, which affects a neurostimulation program selection 1652 or a neurostimulation program modification 1656. For instance, some parameter changes can be implemented by a simple modification to a program operation; other parameter changes may require a new program to be deployed. The results of the parameter or program changes or selection results in definition or adjustment to various stimulation parameters at the neurostimulation stimulation device 1621, causing a different or new stimulation treatment effect 1660.

By way of example, stimulation parameter data 1670 includes operational parameters of the neurostimulation device, which are generated, identified, and/or evaluated by the present systems and techniques can include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The neurostimulator may use current or voltage sources to provide the neurostimulator output and apply any number of control techniques to modify the electrical simulation applied to anatomical sites or systems related to pain or analgesic effect.

In various embodiments, a neurostimulator program can be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

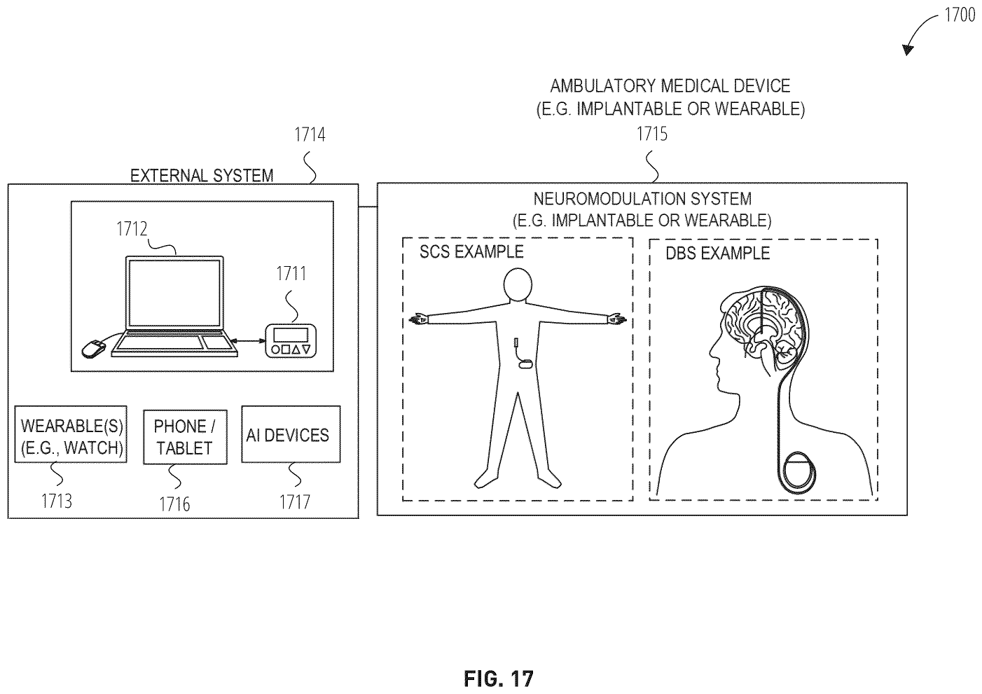

FIG. 17 illustrates, by way of example and not limitation, a block diagram 1700 of the neuromodulation system 1715, for example, like the system depicted and described in connection with FIGS. 12 and 13 can be implemented as a spinal cord stimulation (SCS) system or a deep brain stimulation (DBS) system, example embodiments of the present disclosure can include additional forms of stimulation in a patient. The illustrated neuromodulation system 1715 includes an external system 1714 that can include at least one programming device. The illustrated external system 1714 can include a programmer 1711 configured for use by a clinician, patient, device representative, other caregiver, or a combination thereof to communicate with and program the neuromodulator, and a remote control (not shown) configured for use by the patient to communicate with and program the neuromodulator.

For example, the remote-control device can allow the patient to turn a therapy on and off and/or can allow the patient to adjust patient-programmable parameter(s) of the plurality of modulation parameters. The external system 1714 can further be operatively coupled with one or more patient wearable devices 1713 (e.g., watch, ring, brain-sensing monitor, necklace, heartrate monitor, holter monitor, etc.), a patient computing device 1716 (e.g., phone, computer, tablet, etc.), and/or patient artificial intelligent (AI) devices 1717 (e.g., Amazon® Alexa, Google® Assistant).

FIG. 17 illustrates a medical device as an ambulatory medical device 1715. Examples of ambulatory devices include wearable or implantable neuromodulators. The external system 1714 can include a network of computers, including computer(s) remotely located from the ambulatory medical device 1715 that are capable of communicating via one or more communication networks with the programmer 1711 and/or the remote control. The remotely located computer(s) 1712 and the ambulatory medical device 1715 can be configured to communicate with each other via another external device such as the programmer 1711 or the remote control. The remote-control device and/or the programmer 1711 can allow a user (e.g., patient and/or clinician or device rep) to answer questions as part of the data collection process. The ambulatory medical device may include internal or external sensors that can be used to collect data, which can form at least part of the overall pain data, healthcare-related data, or other patient data to be transferred to a data receiving system. Parameter data for the programmed therapy can form at least part of the healthcare-related data to be transferred to a data receiving system.

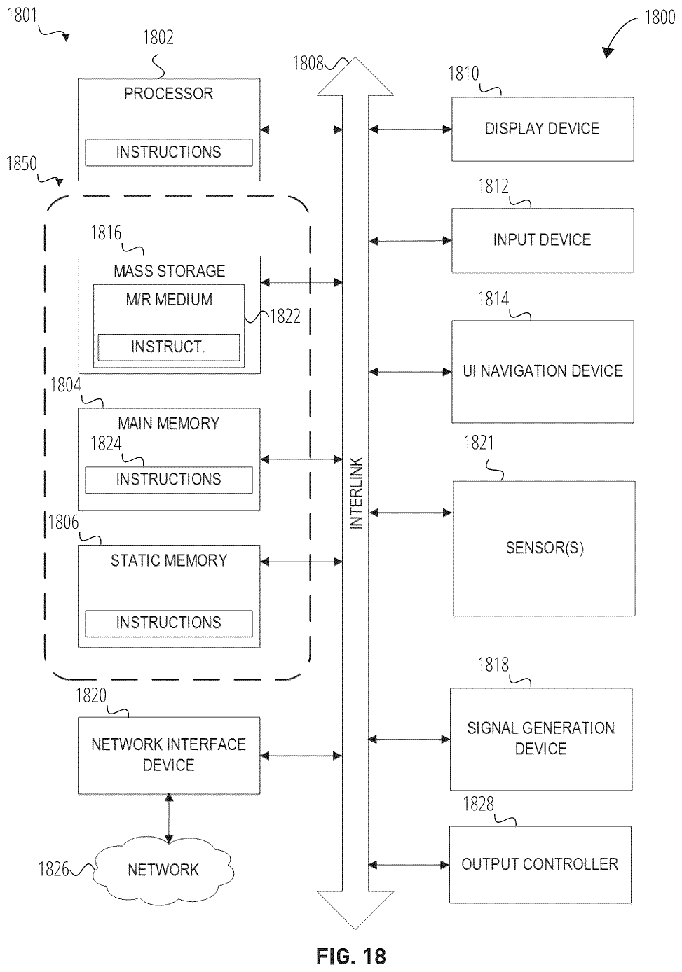

FIG. 18 is a block diagram 1800 illustrating a machine in the example form of a computer system 1801, within which a set or sequence of instructions can be executed to cause the machine to perform any one of the methodologies discussed herein, according to an example embodiment. In alternative embodiments, the machine operates as a standalone device or can be connected (e.g., networked) to other machines.

In a networked deployment, the machine may operate in the capacity of either a server or a client machine in server-client network environments, or it may function as a peer machine in peer-to-peer (or distributed) network environments. The machine can be a personal computer (PC), a tablet PC, a hybrid tablet, a personal digital assistant (PDA), a mobile telephone, an implantable pulse generator (IPG), an external trial stimulator (ETS), an external remote control (RC), a User's Programmer (UP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1801 includes at least one processor 1802 (e.g., a central processing unit (CPU), a graphics processing unit (GPU) or both, processor cores, compute nodes, etc.), a main memory 1804 and a static memory 1806, which communicate with each other via a link 1808 (e.g., bus, interlink). The computer system 1801 can further include a video display unit 1810, an alphanumeric input device 1812 (e.g., a keyboard), and a user interface (UI) navigation device 1814 (e.g., a mouse). In one embodiment, the video display unit 1810, input device 1812 and user interface (UI) navigation device 1814 are incorporated into a touch screen display. The computer system 1801 can additionally include a storage device 1816 (e.g., a drive unit), a signal generation device 1818 (e.g., a speaker), an output controller 1828, a network interface device 1820, and one or more sensors 1821, such as a global positioning system (GPS) sensor, compass, accelerometer, or another sensor. It will be understood that other forms of machines or apparatuses (such as PIG, RC, CP devices, and the like) that are capable of implementing the methodologies discussed in this disclosure may not incorporate or utilize every component depicted in FIG. 18 (such as a GPU, video display unit, keyboard, etc.).

The storage device 1850 includes a mass storage 1816, a machine-readable medium 1822 on which is stored one or more sets of data structures and instructions 1824 (e.g., software) embodying or utilized by any one or more of the methodologies or functions described herein. The instructions 1824 can also reside, completely or at least partially, within the main memory 1804, static memory 1806, and/or within the processor 1802 during execution thereof by the computer system 1801, with the main memory 1804, static memory 1806, and the processor 1802 also constituting machine-readable media.

While the machine-readable medium 1822 is illustrated in an example embodiment to be a single medium, the term “machine-readable medium” can include a single medium or multiple media (e.g., a centralized or distributed database, and/or associated caches and servers) that store the one or more instructions 1824. The term “machine-readable medium” shall also be taken to include any tangible (e.g., non-transitory) medium that is capable of storing, encoding, or carrying instructions for execution by the machine and that cause the machine to perform any one or more of the methodologies of the present disclosure or that is capable of storing, encoding, or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1824 can further be transmitted or received over a communications network 1826 using a transmission medium via the network interface device 1820 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

As used herein, the terms “machine-storage medium,” “device-storage medium,” “machine-readable medium,” and “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media, and/or device-storage media include non-volatile memory, including by way of example semiconductor memory devices, e.g., erasable programmable read-only memory (EPROM), electrically erasable programmable read-only memory (EEPROM), field-programmable gate arrays (FPGAs), and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

The terms “transmission medium” and “signal medium” mean the same thing and may be used interchangeably in this disclosure. The terms “transmission medium” and “signal medium” shall be taken to include any intangible medium that is capable of storing, encoding, or carrying the instructions for execution by the machine depicted in block diagram 1800, and include digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a manner as to encode information in the signal.

The terms “machine-readable medium,” “computer-readable medium,” and “device-readable medium” mean the same thing and may be used interchangeably in this disclosure. The terms are defined to include both machine-storage media and transmission media. Thus, the terms include both storage devices/media and carrier waves/modulated data signals.

The above detailed description is intended to be illustrative, and not restrictive. The scope of the disclosure should, therefore, be determined with references to the appended claims, along with the full scope of equivalents to which such claims are entitled. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method for parameterizing a closed-loop algorithm for providing sub-perception therapy, the method comprising:
delivering neurostimulation;
sensing a neural signal indicative of neural responses, the neural responses each being a response to the delivering of the neurostimulation;
controlling the delivering of the neurostimulation using a plurality of stimulation parameters;
recording evoked compound action potential (ECAP) features received from a sensing circuit;
adjusting stimulation parameters based, at least in part, on the recorded ECAP features in order to maintain consistent neural activation;
recording accelerometer data;
generating a regression model for mapping the accelerometer data to an optimal stimulation parameter to achieve consistent nerve activation, the mapping enabling closed-loop control of the sub-perception therapy;
applying the regression model and a sub-perception dose level to a plurality of sub-perception therapy programs; and
deploying the regression model to a patient via a stimulation output circuit to deliver the neurostimulation.

2. The method of claim 1, further comprising:
identifying a target range for the ECAP features, the target range including an upper value of an ECAP range and a lower value of the ECAP range; and
determining the consistent nerve activation by maintaining values of the ECAP features within the target range.

3. The method of claim 1, further comprising:
prompting the patient to perform one or more physical movements; and
receiving, via a user interface, patient input accounting for environmental factors that impact sensor readings.

4. The method of claim 3, wherein the one or more physical movements include one or more of a plurality of postural changes performed by the patient, the plurality of postural changes including at least one of lying down, standing up, bending over, twisting sidewise, performing daily life activities, and reclining.

5. The method of claim 1, further comprising:
identifying one or more settings in which to suggest alterations for delivering the neurostimulation to the patient, the neurostimulation being at least partially defined based on the accelerometer data.

6. The method of claim 1, further comprising:
identifying one or more settings in which to suggest alterations for delivering the neurostimulation to the patient, the neurostimulation being at least partially defined based on the accelerometer data.

7. The method of claim 1, further comprising:
providing a monitoring platform; and
receiving information on a plurality of accelerometer data, the plurality of accelerometer data representing an overall patient health based on a combination of neurostimulation parameters comprising:
an electrical waveform; and
a selection of electrodes through which the electrical waveform is delivered.

8. The method of claim 7, further comprising:
generating a suggestion for one or more additional combinations of the neurostimulation parameters; and
providing the suggestion to the monitoring platform.

9. The method of claim 1, wherein the plurality of accelerometer data provides supplemental data to the ECAP features to optimize stimulation in a sub-perception domain, and further comprising:
analyzing data for neurostimulation programming.

10. The method of claim 1, further comprising:
initializing the closed-loop algorithm based at least in part on historical patient data; and
adjusting the closed-loop algorithm using patient-specific data.

11. The method of claim 1, further comprising:
recalibrating the mapping to account for changes identified in an implantable pulse generator (IPG), the IPG configured to deliver electrical stimulation through leads and electrodes.

12. The method of claim 1, wherein the regression model is further configured to generate a customized mapping for each patient by collecting ECAP and accelerometer data in a clinical setting, and wherein the regression model collects ECAP factors during the mapping, the ECAP factors selected from at least one of an amplitude, a detection threshold, a perception level, a linearity with stimulation intensity, the stimulation parameters, an electrode location, or neural activation levels.

13. A machine-storage medium embodying instructions that, when executed by a machine, cause the machine to perform operations comprising:
delivering neurostimulation;
sensing a neural signal indicative of neural responses, the neural responses each being a response to the delivering of the neurostimulation;
controlling the delivering of the neurostimulation using a plurality of stimulation parameters;
recording evoked compound action potential (ECAP) features received from a sensing circuit;
adjusting stimulation parameters based, at least in part, on the recorded ECAP features in order to maintain consistent neural activation;
recording accelerometer data;
generating a regression model for mapping the accelerometer data to an optimal stimulation parameter to achieve consistent nerve activation, the mapping enabling closed-loop control of a sub-perception therapy;
applying the regression model and a sub-perception dose level to a plurality of sub-perception therapy programs; and
deploying the regression model to a patient via a stimulation output circuit to deliver the neurostimulation.

14. The machine-storage medium embodying the instructions of claim 13, further comprising:
identifying a target range for the ECAP features, the target range including an upper value of an ECAP range and a lower value of the ECAP range; and
determining the consistent nerve activation by maintaining values of the ECAP features within the target range.

15. The machine-storage medium embodying the instructions of claim 13, further comprising:
prompting the patient to perform one or more physical movements; and
receiving, via a user interface, patient input accounting for environmental factors that impact sensor readings.

16. The machine-storage medium embodying the instructions of claim 15, wherein the one or more physical movements include one or more of a plurality of postural changes performed by the patient, the plurality of postural changes including at least one of lying down, standing up, bending over, twisting sidewise, performing daily life activities, and reclining.

17. The machine-storage medium embodying the instructions of claim 13, further comprising:
identifying one or more settings in which to suggest alterations for delivering the neurostimulation to the patient, the neurostimulation being at least partially defined based on the accelerometer data.

18. The machine-storage medium embodying the instructions of claim 13, further comprising:
providing a monitoring platform; and
receiving information on a plurality of accelerometer data, the plurality of accelerometer data representing an overall patient health based on a combination of neurostimulation parameters comprising:
an electrical waveform; and
a selection of electrodes through which the electrical waveform is delivered.

19. The machine-storage medium embodying the instructions of claim 18, further comprising:
generating a suggestion for one or more additional combinations of the neurostimulation parameters; and
providing the suggestion to the monitoring platform.

20. A system for parameterizing a model for providing sub-perception therapy, the system comprising:
one or more hardware processors of a machine; and
at least one memory storing instructions that, when executed by the one or more hardware processors, cause the system to perform operations comprising:
recording evoked compound action potential (ECAP) features received from a sensing circuit;
adjusting stimulation parameters based, at least in part, on the recorded ECAP features in order to maintain consistent neural activation;
recording accelerometer data;
generating a regression model for mapping the accelerometer data to an optimal stimulation parameter to achieve consistent nerve activation, the mapping enabling closed-loop control of the sub-perception therapy;
applying the regression model and a sub-perception dose level to a plurality of sub-perception therapy programs; and
deploying the regression model to a patient via a stimulation output circuit to deliver neurostimulation.
